(19) United States
(12) Patent Application Publication
(10) Pub. No.: US 2025/0205316 A1
(43) Pub. Date: Jun. 26, 2025

(54) COLLOIDAL FIBRIN NANOPARTICLE COMPOSITIONS AND USES

(71) Applicant: NORTH CAROLINA STATE UNIVERSITY, Raleigh, NC (US)

(72) Inventors: Ashley C. Brown, Raleigh, NC (US); Nina Moiseiwitsch, Raleigh, NC (US); Sanika Pandit, Raleigh, NC (US); Kimberly A. Nellenbach, Raleigh, NC (US); Lauren Schnabel, Raleigh, NC (US); Shannon Connard, Raleigh, NC (US); Alexa Kuyvenhoven, Raleigh, NC (US)

(21) Appl. No.: 18/921,390

(22) Filed: Oct. 21, 2024

Related U.S. Application Data
(60) Provisional application No. 63/613,160, filed on Dec. 21, 2023.

(51) Int. Cl. A61K 38/48; A61K 33/06; A61K 35/16; A61K 35/19; A61K 38/36; A61K 45/06

(52) U.S. Cl. CPC A61K 38/4833; A61K 33/06; A61K 35/16; A61K 35/19; A61K 38/363; A61K 45/06

(57) ABSTRACT

Disclosed herein are colloidal mixtures including fibrin nanoparticles. The mixtures are useful to treat wounds and promote healing. The mixtures can also include a blood co-factor, thrombin, and a calcium salt.

This application claims the benefit of U.S. Provisional Application 63/613,160, filed Dec. 21, 2023, the contents of which are hereby incorporated in its entirety.

### STATEMENT OF GOVERNMENT SUPPORT

This invention was made with government support under grant number CBET2211404 awarded by the National Science Foundation and grant number HL163869 awarded by the National Institutes of Health. The government has certain rights in the invention.

### BACKGROUND

Scaffold porosity is of critical importance for cellular infiltration and tissue regeneration throughout the body. Porosity, however, is hard to control in the lab synthesis of naturally-derived scaffolds, such as collagen and chitosan. Further, material void space often comes as a trade-off with material strength. The advent of microporous annealed particle (MAP) hydrogels has led to an altogether new level of control over final material porosity when building scaffolds. In MAP gels, particle size and concentration may be adjusted as desired to achieve optimal porosity and void volume for different applications. As these are formed from pre-polymerized particles and delivered in a liquid or gel suspension, they are very convenient for both topical and injectable uses. Upon localized delivery, the particle slurry is then annealed via light-based or chemical crosslinking. However, it is known that the extracellular matrix (ECM) is an essential aspect of cellular regeneration in vivo, and MAP gels do not capture the fibrillar characteristics of the ECM, nor do they benefit from the added material strength offered by a fibrillar network. Native ECM materials in the body are often soft materials, but their micro level fibrillar structure provide crucial structural cues to promote and guide cell growth across a multitude of tissues, including tendon, muscle, nerve, and skin. We were inspired by the interplay between soft colloids and microfibrillar structure in the body to investigate the impact of different relative compositions of these two material types and develop biomimetic wound healing materials. Our goal is to bring together the porosity, tunability, and convenience of MAP hydrogel scaffolds and the benefits of a fibrillar matrix to develop a surgical sealant with superior healing capabilities for potential use both inside and outside of the body.

Fibrin, which forms the structural support for blood clots, is an example of a fibrillar material of crucial importance to wound healing throughout the body. In the final steps of the coagulation cascade, the serine protease thrombin cleaves soluble fibrinogen into insoluble fibrin, which forms the scaffold for cellular infiltration into the wound area. Bulk fibrin gels are commonly used for clinical applications. While a number of different surgical sealants exist, the only ones that are currently approved for use within the body are high density fibrin glues. These glues, which are naturally-derived, work by mixing two concentrated solutions of fibrinogen and thrombin (80-120 mg/mL and 300-600 U/mL, respectively, compared to physiologic concentrations of 2-4 mg/mL and 0.25-1 U/mL), mimicking the final steps of the coagulation cascade through the mixing of these two components. However, the high concentrations of thrombin and fibrinogen, more than 40-100× physiologic levels, create short working-times for clinicians and a resultant hyperdense structure which leads to poor cellular infiltration during healing. This is of particular concern with tissues where healing times are already very long, including neurons and vascular networks. Furthermore, current high-density fibrin glues (HDFGs) require refrigeration, forcing thaw times and cold chain requirements, which make their use impractical in emergencies or spaces where freezer maintenance is impossible, as in rural or temporary hospitals.

Non-healing wounds represent a significant clinical problem in both human and veterinary medicine. Patients affected by non-healing wounds often have decreased mobility and an increased risk of death. Moreover, chronic wounds have also been identified in the veterinary space, particularly in equine distal limbs.

There remains a need for improved systems and methods of treating wounds, including non-healing wounds. There remains a need for improved sealant compositions to treat wounds and promote healing.

### BRIEF DESCRIPTION OF THE FIGURES

FIG. 1 depicts an overview of fibrin-based nanoparticles (FBNs) and FBN Gels. (A) Schematic comparing material properties of high-density fibrin glues with those of FBN gels. (B) Representative cryoSEM images of high-density fibrin glue, FBN gel, and a fibrin clot of physiologic density. Scale bar=10 μm. (C) Representative confocal images of HUVEC cells seeded in either high density fibrin glue or FBN gel, after one week of growth, demonstrating relative cellular-level porosity. Scale bar=200 μm. (D) Schematic of fibrin-based nanoparticle production. (E) Representative atomic force microscopy image of fibrin-based nanoparticle. Scale bar=1 μm. (F) Representative image of lyophilized FBNs. Scale bar=1 cm. (G) Representative images before and after rapid resuspension of lyophilized FBNs in deionized water via 10 seconds of mixing via vortex.

FIG. 2 depicts tests between whole blood gels, high-density fibrin glue (HDFG) and whole blood-FBN gels of varying FBN concentration. (A) Average polymerization curves determined via rheometry for whole blood clots, high density fibrin glue, and whole blood-FBN gels using differing concentrations of FBNs. Thrombin (0.5 U/mL) and CaCl₂ (0.05 mM) and FBNs (varying final concentrations) were added to whole blood. (B) Comparison of gelation time between whole blood clots, high density fibrin glue, and whole blood-FBN gels with differing concentrations of FBNs. (C) Comparison of storage modulus after 15 minutes of polymerization between whole blood clots, high density fibrin glue, and whole blood-FBN gels with differing concentrations of FBNs. (D) Schematic demonstrating Instron UTS methods utilized to measure tension, shear adhesion, and wound closure. (E) Comparison of tensile strength between gels. (F) Comparison of shear adhesive strength between gels. (G) Comparison of wound closure rupture extension between gels. (H) Schematic of material leak testing process. (l) Comparison of leak pressure between biomaterials. (J) Comparison of degradation over four hours between gels. (K) Comparison of tensile strength of whole blood-FBN gel (330M FBN/mL) made using newly polymerized FBNs or 1-year old FBNs (lyophilized; room temperature (RT) storage). (L) Comparison of adhesive strength of whole blood-FBN gel (330M FBN/mL) made using newly polymerized FBNs or 1-year old FBNs (lyophilized; RT storage). (M) Comparison of leak pressure of whole blood-FBN gel (330M FBN/mL) made using newly polymerized FBNs or 1-year old FBNs (lyophilized; RT storage). ns=p>0.05, *=p<0.05, **=p<0.01, ***=p<0.001

FIG. 3 depicts differences in FBN gel structure and mathematical modeling in response to particle concentration. (A) Schematic describing how increasing FBN concentration affects fibrin-colloid composite structure. (B) Representative cryoSEM images of a PPP gel, as well as PPP-FBN gels with 66M, 330M, and 530M FBN/mL. Scale bar=10 μm. (C) Representative detailed cryoSEM images of FBNs integrated into the fibrin matrix (top image) and FBNs polymerized among themselves to form highly aligned secondary fibrin network (bottom image). FBNs false colored in blue. Scale bar=1 μm. (D) Comparison of fiber thickness between PPP gels and PPP-FBN gels with different concentrations of FBNs. (E) Comparison of porosity between PPP gels and PPP-FBN gels with different concentrations of FBNs. (F) Comparison of fiber alignment between PPP gels and PPP-FBN gels with different concentrations of FBNs. Measurements were taken based on cryoSEM images taken. (G) Schematic of Instron UTS method used to generate stress-relaxation curves for mathematical modeling. (H) Stress-relaxation curves from high-density fibrin glue (HDFG), differentially colored in red and blue to draw attention to two sub-populations of curve patterns. Average curves for each sub-population included in black. N=10 total; 5 per subpopulation. (I) Average stress-relaxation curves for whole blood clots, WB-FBN gels with 66M, 130M, 260M, or 400M FBN/mL, and HDFG sub-populations plotted together (left) with detail inset (right). For 400M FBN/mL group, representative curve was plotted, rather than average, to preserve depiction of inertial regime in this group. (J) Schematic depicting stress-relaxation curve changes as FBNs are added to whole blood, and FBN concentration is increased, in accordance with (K) Representative stress-relaxation curves for whole blood clots and WB-FBN gels (particle concentrations: 66M, 130M, 260M & 400M FBN/mL; these same concentrations are compared throughout remaining panels.) plotted alongside their best-fit Kelvin mathematical model, where appropriate. Kelvin schematic included for each representative image to depict system differences from baseline whole blood gel without FBNs. (L) Comparison of stress-relaxation time between whole blood clots, whole blood-FBN gels, and HDFG. (M) Comparison of strain-relaxation time between whole blood clots, whole blood-FBN gels, and HDFG. (N) Comparison of equilibrium stress between whole blood clots, whole blood-FBN gels, and HDFG. *=p<0.05, **=p<0.01, ***=p<0.001, ****=p<0.0001.

FIG. 4 depicts an in vivo evaluation of FBN gels for wound healing in a leporine model of vascular anastomosis. (A) Schematic of leporine vascular healing assay employed. (B) Representative images of FBN gel following polymerization in situ on rabbit vessel injury site (top) and injury site with stitches alone (bottom). (C) Representative ultrasound images used to evaluate patency of vessel surgical site before assay endpoint. Red arrows indicate location of vessel cross-section. Narrowing can be seen at the surgical site. (D) Thrombogenicity determined via aPTT clotting time of whole blood in the presence of the clot, FBN gel, or HDFG. (E) Comparison of vessel cross-sectional area between surgical groups: stitches alone, stitches with high-density fibrin glue, stitches with FBN gel, and stitches with tazarotene-loaded FBN (tFBN) gel. Vessels that experienced complete thrombosis are highlighted in red. (F) Representative H&E histology from each surgical group, with wound area circumscribed in yellow and detailed higher magnification inset for each wound site. Scale bar=400 μm. (G) Comparison of wound percentage of vessel area between surgical groups: stitches alone, stitches with high-density fibrin glue, stitches with FBN gel, and stitches with tazarotene-loaded FBN (tFBN) gel. Vessels that experienced complete thrombosis were excluded from this comparison. (H) Comparison of endothelial continuity between surgical groups: stitches alone, stitches with high-density fibrin glue, stitches with FBN gel, and stitches with tazarotene-loaded FBN (tFBN) gel. (I) Comparison of vessel thickness between surgical groups: stitches alone, stitches with high-density fibrin glue, stitches with FBN gel, and stitches with tazarotene-loaded FBN (tFBN) gel. Vessels that experienced complete thrombosis were excluded from this comparison. (J) Comparison of endothelial hypertrophy between surgical groups: stitches alone, stitches with high-density fibrin glue, stitches with FBN gel, and stitches with tazarotene-loaded FBN (tFBN) gel. (K) Representative fibrin immunohistochemistry (IHC) images of vessels treated with FBN gel (left) and with high-density fibrin gel (right). Fibrin is in red, allowing for visualization of FBNs and HDFG remnants. Cell nuclei are in blue. Stitches, where present, are false colored in green. Scale bar=400 μm. (L) Comparison of fibrin integrated density at vessel injury site between surgical groups via analysis of fibrin IHC images. (M) Comparison of adhesion grading of vessel surgical site, determined during tissue collection. Grade 0=no attachment of vessel to surrounding tissue. Grade 5=full circumferential attachment to surrounding tissue. *=p<0.05, **=p<0.01, ***=p<0.001, ****=p<0.0001

FIG. 5 depicts a comparison of transcriptomic and cellular outcomes of colloidal-fibrillar and bulk sealant materials. (A) Heatmap comparison of transcriptomic analysis of vessel wound site tissue between surgical groups: stitches alone, stitches with high-density fibrin glue, stitches with FBN gel, and stitches with tazarotene-loaded FBN (tFBN) gel. (B) Schematic representation of vasculogenesis assay. HUVEC and HLF cells were seeded in the biomaterial (HDFG, FBN gel, or tFBN gel) during in situ polymerization of the material within the central channel. (C) Representative images of vascular networks formed during vasculogenesis assay. HLFs are stained in green, HUVECs are stained in red, and cell nuclei are cyan. Scale bar=100 μm. (D) Detailed representative images of HUVEC networks formed during vasculogenesis assay and comparison of total HUVEC cellular area in vascular network between FBN gel, tFBN gel, and HDFG. Scale bar=100 μm. (E) Detailed representative images of HLF networks formed during vasculogenesis assay and comparison of total HLF cellular area in vascular network formed between FBN gel, tFBN gel, and HDFG. N=5-7 Scale bar=200 μm. (F) Schematic of HUVEC thin film cellular effects assay. Thin films of biomaterial (FBN gel, tFBN gel, or HDFG) were allowed to polymerize for 2 hr. HUVECs were seeded onto thin films and given normal HUVEC media. Cells were counted and imaged after 16 hours. (G) Representative images of HUVECs after culture on FBN gel, tFBN gel, or HDFG for 16 hours. Scale bar=100 μm. (H) Comparison of HUVEC cellular attachment after 16 hours of culture on FBN gel, tFBN gel, or HDFG. (I) Comparison of HUVEC individual cell area after 16 hours of culture on FBN gel, tFBN gel, or HDFG. (J) Comparison of HUVEC individual cell perimeter after 16 hours of culture on FBN gel, tFBN gel, or HDFG. (K) Comparison of HUVEC individual cell circularity after 16 hours of culture on FBN gel, tFBN gel, or HDFG. *=p<0.05, **=p<0.01, ***=p<0.001, ****=p<0.0001.

FIG. 6 depicts (A) FBN size histogram, as calculated using NanoSight Particle Tracking Technology. (B) Representative FBN image captured using atomic force microscopy.

FIG. 7 depicts comparison of FBN drug loading by initial bulk gel concentration

FIG. 8 depicts a sample data curve of wound closure test.

FIG. 9 depicts additional FBN Gel and HDFG Structural Analysis. (A) Representative confocal images of FBN gels of different FBN concentrations. Scale bar=50 μm. (B) Comparison of fiber density by FBN concentration. (C) Representative gross image of HDFG (outlined in orange) with visible opacities and transparencies, indicative of non-homogeneous polymerization.

FIG. 10 depicts a Kelvin Model Fit and Inertial Noise. (A) Representative data demonstrating inertial noise in whole blood gels with 260 million FBNs/mL. (B) Kelvin model fits to average data curves with respective residuals plotted below. (C) Kelvin model mechanistic diagram.

FIG. 11 depicts a Kelvin Model Fit for HDFG and Extended Kelvin Model (A) Kelvin model fits to average HDFG data curves with respective residuals plotted below. (B) Extended Kelvin model mechanistic diagram.

FIG. 12 depicts tazarotene effects on vascular network formation. (A) Schematic of vasculogenesis assay used. (B) Representative confocal images of vascular networks grown from human lung fibroblasts (HLF) and human umbilical vein endothelial cells (HUVEC) in the presence of free tazarotene of different concentrations. Scale bar=100 μm. (C) Representative confocal images from (B) with DAPI removed for better visualization of the endothelial network. Scale bar=100 μm. (D) Comparison of endothelial cell area in vascular networks across tazarotene dose exposure. (E) Comparison of average segment width in vascular networks across tazarotene dose exposure. (F) Comparison of average segment length in vascular networks across tazarotene dose exposure. (G) Comparison of number of branch points in vascular networks across tazarotene dose exposure.

FIG. 13 depicts tazarotene effects on HUVEC morphology and attachment. (A) Schematic of bulk drug delivery via fibrin gel. (B) Representative images of HUVECs grown in the presence of bulk-delivered tazarotene of different concentrations. Scale bar=100 μm. (C) Comparison of endothelial cell attachment across tazarotene dose exposure. (D) Comparison of endothelial cell area across tazarotene dose exposure. (E) Comparison of endothelial cell circularity across tazarotene dose exposure. (F) Comparison of endothelial cell perimeter across tazarotene dose exposure.

FIG. 14 depicts effects of nanoparticle delivery of tazarotene on HUVEC morphology and attachment. (A) Schematic of FBN drug loading via polymerization. (B) Representative images of HUVECs grown in the presence of nanoparticle-delivered tazarotene of different concentrations. Scale bar=100 μm. (C) Comparison of endothelial cell area across nanoparticle tazarotene dose exposure. (D) Comparison of endothelial cell attachment across nanoparticle tazarotene dose exposure. (E) Comparison of endothelial cell circularity across nanoparticle tazarotene dose exposure. (F) Comparison of endothelial cell perimeter across nanoparticle tazarotene dose exposure.

FIG. 15 depicts Surgical Vessel Thickness, Including Control Values

FIG. 16 depicts Surgical Use of HDFG Results in Retained Mass at 3 Weeks. (A) Representative image of HDFG remnant retained at 3 weeks after surgery. Scale bar=0.5 cm (B) Representative image of retained HDFG and capsule formation as seen during tissue collection. (C) Fibrin IHC of retained mass confirms that it is fibrin. Scale bar=400 am (D) Comparison of 24-hour degradation of mass between whole blood gels, FBN gels, and HDFG.

FIG. 17 depicts results from Transcriptomic Analysis of Tissue Collected From In Vivo Studies. All expressed as percentage difference in expression compared to healthy vessel. (A) Comparison of collagen expression. (B) Comparison of inflammatory marker expression. (C) Comparison of TGF-β1 expression. (D) Comparison of VCAM1 expression. *=p<0.05.

FIG. 18 depicts the self-assembly of fibrin nano-particles in different HEPES concentration: (A) 1×HEPES (B) 0.25×HEPES (C) 0.1×HEPES (D) 0.05×HEPES (E) No HEPES.

FIG. 19 depicts giant fibrin nanoparticles (gFBN) produced by varying sonication levels: (A) tFBN have an average dry diameter of 230-250 nm (B) gFBN have an average dry diameter of 640-1,000 nm.

FIG. 20 depicts that equine plasma can be used to make fibrin nanoparticles (eFBN) (A) shows eFBN are similar in size to human fibrinogen FBNs. (B) shows clotting time determined from APTT. (C) shows eFBN impact clot structure.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

As used herein, “platelet-poor plasma” refers to plasma samples having less than 10×10³ platelets per microliter of plasma. As used herein, “platelet-rich plasma” refers to plasma samples having greater than 10×10³ platelets per microliter of plasma.

As used herein, a “blood co-factor” is a blood component that includes fibrinogen. This can include whole blood, plasma, or isolated fibrinogen.

As used herein, “neonatal fibrin” refers to fibrin that has been obtained from blood collected from organisms within the first few months of life, including blood obtained from the umbilical cord. This fibrinogen will have at least double the sialic acid content compared to healthy adult fibrinogen from the same specifies.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Compounds disclosed herein may be provided in the form of acceptable salts. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate.

Disclosed herein are colloidal mixtures that include fibrin nanoparticles, a blood co-factor, thrombin, and a calcium salt.

In certain implementations the blood co-factor can be whole blood, plasma, purified fibrinogen, or a combination thereof. In certain implementations the plasma can be platelet-poor plasma, platelet-rich plasma.

In certain implementations the calcium salt can be CaCl₂.

In certain implementations the calcium salt is present in the colloidal mixture at a concentration from 1-1,000 μM, from 5-1,000 μM, from 10-1,000 μM, from 25-1,000 μM, from 50-1,000 μM, from 100-1,000 μM, from 250-1,000 μM, from 1-500 μM, from 5-500 μM, from 10-500 μM, from 25-500 μM, from 50-500 μM, from 100-500 μM, from 250-500 μM, from 1-250 μM, from 5-250 μM, from 10-250 μM, from 25-250 μM, from 50-250 μM, from 100-250 μM, from 1-100 μM, from 5-100 μM, from 10-100 μM, from 25-100 μM, from 50-100 μM, from 1-10 μM, from 1-25 μM, from 5-50 μM, or from 25-75 μM.

In certain implementations the thrombin is present in the colloidal mixture at a concentration from 0.01-10 U/ml, from 0.05-10 U/ml, from 0.1-10 U/ml, from 0.5-10 U/ml, from 1-10 U/ml, from 0.01-2.5 U/ml, from 0.05-2.5 U/ml, from 0.1-2.5 U/ml, from 0.5-2.5 U/ml, from 1-2.5 U/ml, from 0.01-1.5 U/ml, from 0.05-1.5 U/ml, from 0.1-1.5 U/ml, from 0.5-1.5 U/ml, from 0.01-1 U/ml, from 0.1-1 U/ml, from 0.5-1 U/ml, from 0.01-0.75 U/ml, from 0.05-0.75 U/ml, from 0.1-0.75 U/ml, or from 0.25-0.75 U/ml.

In certain implementations the fibrin nanoparticles can be obtained from neonatal fibrin. In certain implementations the fibrin nanoparticles can be obtained from cord blood fibrin. In some instances, the fibrin nanoparticles can be obtained from human fibrin. In certain implementations, the fibrin nanoparticles can be obtained from a domestic animal, for example a horse or a ruminant such as a cow, goat, or sheep.

In certain implementations the fibrin nanoparticles are present in the colloidal mixture at a concentration from 0.1-250 mg/ml, from 1-250 mg/ml, from 5-250 mg/ml, from 10-250 mg/ml, from 25-250 mg/ml, from 50-250 mg/ml, from 100-250 mg/ml, from 1-100 mg/ml, from 5-100 mg/ml, from 10-100 mg/ml, from 25-100 mg/ml, from 50-100 mg/ml, from 1-75 mg/ml, from 5-75 mg/ml, from 10-75 mg/ml, from 25-75 mg/ml, from 1-50 mg/ml, from 5-50 mg/ml, from 10-50 mg/ml, from 25-50 mg/ml, from 1-25 mg/ml, from 5-25 mg/ml, from 10-25 mg/ml, or from 20-40 mg/ml.

In certain implementations the fibrin nanoparticles are present in the colloidal mixture in an amount of 1-5,000M particles/ml, 1-1,000M particles/ml, 25-1,000M particles/ml, 50-1,000M particles/ml, 100-1,000M particles/ml, 250-1,000M particles/ml, 500-1,000M particles/ml, 1-500M particles/ml, 25-500M particles/ml, 50-500M particles/ml, 100-500M particles/ml, 250-500M particles/ml, 1-250M particles/ml, 25-250M particles/ml, 50-250M particles/ml, 100-250M particles/ml, 1-100M particles/ml, 25-100M particles/ml, 50-100M particles/ml, 1-50M particles/ml, 1-25M particles/ml, or 25-50M particles/ml.

In certain implementations the fibrin nanoparticles have an average hydrodynamic (dry) diameter from 50-1,000 nm, from 50-500 nm, from 50-250 nm, from 100-500 nm, from 100-250 nm, from 100-300 nm, from 200-300 nm, from 250-350 nm, from 300-400 nm, from 250-500 nm, or from 500-1,000 nm.

In certain implementations the colloidal mixture can include a therapeutic agent. Exemplary therapeutic agents include antimicrobials, analgesics, growth factors, cell therapies, small molecules and organoids. Anti-microbial agents are agent that inhibit the growth or kill microbes such as virus, bacteria, fungi, and parasites. Anti-microbial agents include anti-bacterial agents, anti-mycobacterial agents, anti-viral agents, anti-fungal agents, and anti-parasite agents. Suitable antimicrobials include antibiotics, antimicrobial peptides and metallic compounds.

Suitable antibiotics include penicillins, cephalosporins, quinolones (including fluoroquinolones), aminoglycosides, monobactams, carbapenems, tetracyclines, macrolides, peptides, and others. In some embodiments, the antibiotics is one or more compounds selected from streptomycin, neomycin, kanamycin, amikacin, gentamycin, tobramycin, sisomicin, arbekacin, apramycin, netilmicin, paromomycin, spectinomycin, ciprofloxacin, levofloxacin, lomefloxacin, moxifloxacin, norfloxacin, ofloxacin, sparfloxacin, trvafloxacin, gatifloxacin, gemifloxacin, cinoxacin, nalidixic acid, azithromycin, clarithromycin, dirithromycin, erythromycin, roxithromycin, troleandomycin, telithromycin, spectinomycin, indolicidin, defensin, cecropin, magainin, vancomycin, teicoplanin, telavancin, ramoplanin, decaplanin, bleomycin, colistin (polymyxin E), colistin A (polymyxin E1), colistin B (polymyxin E2), colistin sulfate, colistimethate sodium, actinomycin, bacitracin, polymyxin B, gentamicin, gentamicin sulfate, neomycin, kanamycin, tobramycin, metronidazole, clotrimazole, secnidazole, ornidazole, tinidazole, linezolid, doxycycline, tetracycline, oxytetracycline, chlortetracycline, demeclocycline, lymecycline, meclocycline, methacycline, minocycline, rolitetracycline, and tigecycline.

Suitable analgesics include opioids, capsaicin, diclofenac, lidocaine, benzocaine, methyl salicylate, trolamine, prilocaine, pramoxine, dibucaine, phenol, tetracaine, camphor, dyclonine, and menthol. Suitable anti-inflammatories include alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lornoxicam, loteprednol etabonate, meclofenamate sodium, meclofenamic acid, meclorisone dibutyrate, mefenamic acid, mesalamine, meseclazone, methylprednisolone suleptanate, morniflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, and zomepirac sodium.

In certain implementations the therapeutic agent can be a retinoid, for example the therapeutic agent can be tazarotene.

In certain implementations the growth factor can be keratinocyte growth factor (KGF), platelet derived growth factor (PDGF), transforming growth factor-beta (TGFβ), interleukin, activin, colony stimulating factor, connective tissue growth factor (CTGF), epidermal growth factor (EGF), Epigen, erythropoietin, fibroblast growth factor (FGF), galectin, hepatoma-derived growth factor (HDGF), hepatocyte growth factor, insulin-like growth factor binding protein (IGFBP), insulin-like growth factor, insulin, leptin, macrophage migration inhibitory factor, melanoma inhibitory factor, myostatin, noggin, nephroblastoma overexpressed (NOV), omentin, oncostatinM, osteopontin, osteoprotogerin (OPG), periostin, placenta growth factor, placental lactogen, prolactin, RANK ligand, retinol binding protein, stem cell factor, transforming growth factor, vascular endothelial growth factor (VEGF), or a combination thereof.

In certain implementations the cell therapy can be a stem cell, T cell, a lymphocyte, a natural killer cell, bone marrow cell, hepatocyte, fibroblast, pancreatic islet cell, chondrocyte, keratinocyte, dendritic cell, macrophage, or combination thereof.

Also disclosed are methods of making the colloidal mixture disclosed herein, which include the step combining fibrin nanoparticles with thrombin and a blood co-factor. In certain implementations the colloidal mixture is formed by combining lyophilized fibrin nanoparticles with composition that includes thrombin and a blood co-factor.

In certain implementations, fibrin nanoparticles may be obtained by polymerizing fibrinogen with thrombin to obtain a polymerized fibrin, and then shearing the polymerized fibrin to obtain fibrin nanoparticles. In certain implementations, the polymerized fibrin is sheared by exposure to ultrasonication. In certain implementations, the polymerized fibrin can be passed through one or more filters having a specified pore size, typically from 1-100 μm. The filtered product can be lyophilized using conventional techniques.

In some implementations neonatal fibrinogen can be polymerized with thrombin to obtain a polymerized fibrin. In certain implementations cord blood fibrinogen can be polymerized with thrombin to obtain a polymerized fibrin. In certain implementations cord blood. In some implementations the fibrinogen nanoparticles is human fibrinogen. In certain implementations, the fibrinogen is obtained from a domestic animal, for example a horse or a ruminant such as a cow, goat, or sheep.

In some implementations the fibrinogen is polymerized with thrombin in a buffered solution. In some implementations the fibrinogen is polymerized with thrombin in an aqueous solution comprising N-2-hydroxyethylpiperazine-N′-2-ethanesulfonic acid (“HEPES”). The concentration of the HEPES can be from 0.1-100 mM, from 1-100 mM, from 10-100 mM, from 10-50 mM, from 50-100 mM, from 1-10 mM, from 1-5 mM, from 5-10 mM, from 10-25 mM, or from 25-50 mM.

Following polymerization, the polymerized fibrin can be passed through a sieve or filter to obtain particles of a desired size. In some implementations, the polymerized fibrin can be passed through a filter having a pore size from 1-250 μm, from 1-100 μm, from 10-100 μm, from 25-100 μm, from 50-100 μm, from 50-150 μm, from 100-250 μm, from 10-50 μm, or from 100-150 μm.

In some implementations, the polymerized fibrin is sonicated. In some implementations the particle size can be controlled by varying the sonication level. In some implementations, the sonicator can be a Branson Sonifier Cell Disruptor, for example model 200 or 550. If smaller nanoparticles are desired, the SFX550 can be used with ampliture=10%, on 10 ms, off 50 ms, for total on time of 15 s. Larger nanoparticles can be obtained using reduced output levels for shorter periods of time.

In some implementations, the polymerized fibrin is filtered and then sonicated.

In some implementations, the polymerized fibrin is sonicated then filtered. In further implementations, the polymerized fibrin is filtered, then sonicated, then filtered again. In certain implementations, the second filtration can be performed with a filter having a pore size that is from 10-90%, 25-75%, 10-50%, or 50-90% the pore size used for the first filtration.

In some implementation, a calcium salt is combined with the fibrin nanoparticles, thrombin, and blood co-factor. In certain implementations the calcium salt is combined with the lyophilized fibrin nanoparticles prior to mixing with the thrombin and co-factor. In some implementations, the calcium salt is combined with the thrombin and co-factor prior to mixing with the fibrin nanoparticles. In further implementations, a calcium salt can be combined with each of the fibrin nanoparticles and co-factor prior to mixing. In certain implementations, the calcium salt is calcium chloride.

In some instances, lyophilized fibrin nanoparticles can be reconstituted in an aqueous solution that includes a calcium salt, for example calcium chloride. The aqueous solution can be sterile water or a physiologically suitable buffer. The reconstituted nanoparticles can then be combined with the blood co-factor.

In certain implementations the blood co-factor can be obtained from the subject that will receive the colloidal mixture. The subject's blood can be withdrawn using conventional techniques, and in some embodiments directly combined with the fibrin nanoparticles, either lyophilized nanoparticles or nanoparticles in aqueous solution. In certain implementations, the plasma may be separated from the withdrawn blood and used as the co-factor.

The colloidal mixtures disclosed herein may be used to treat a wound in a subject in need thereof. In certain implementations, the colloidal mixture may be applied directly to the surface of the wound. Blood may be obtained from the subject, either from the area of the wound or from a non-wounded region, and then combined with nanoparticles to form the colloidal mixture that is applied to the wound.

In certain implementations, the colloidal mixture can be used to treat a surgical wound, a chronic wound, or a trauma wound. The colloidal mixture can be used to repair damaged vascular tissue, damaged ocular tissue, or damaged gastrointestinal tissue. In certain implementations the colloidal mixture can be used to promote healing following a surgical anastomosis. In certain implementations the colloidal mixture can be used to promote healing following a vascular anastomosis. In certain implementations the colloidal mixture can be used to promote healing following an intestinal anastomosis. Two or more vessels may be joined together and the colloidal mixture is applied to the region where they contact one another. In certain implementations the colloidal mixture may be applied to one or more vessels to be joined prior to the anastomotic procedure. Following anastomosis, a colloidal mixture (either the same or different) may be applied to the joined region.

Also disclosed herein are kits suitable for forming the colloidal mixtures disclosed herein. In certain implementations the kit may include fibrin nanoparticles, a calcium salt, and thrombin. In certain implementations the kit may further include a blood co-factor. In some implementations the kit may include a blood collecting system suitable for obtaining a blood co-factor from a subject. In certain implementations the kit can include lyophilized fibrin nanoparticles in a sterile container. The container may further include one or more therapeutic agents and/or calcium salts.

### EXAMPLES

The following examples are for the purpose of illustration of the invention only and are not intended to limit the scope of the present invention in any manner whatsoever.

### Example 1

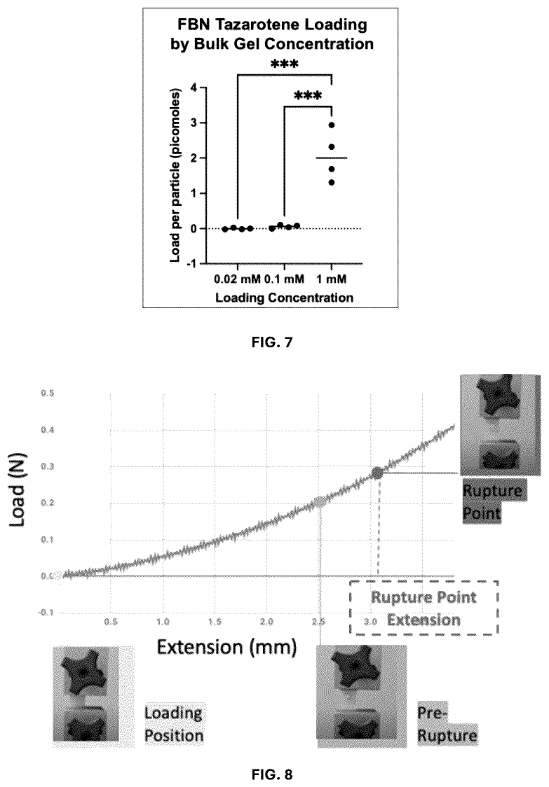

A 5 mL bulk fibrin gel is produced to a final concentration of 0.5 U/mL thrombin, 2 mg/mL fibrinogen, and 25 mM N-2-hydroxyethylpiperazine-N′-2-ethanesulfonic acid (HEPES) buffer (0.15M NaCl, 5 mM CaCl₂, pH 7.4). After allowing two hours for polymerization, the bulk gel is diced and pushed through a 70 μm filter. The resultant liquid is pulse-sonicated on ice for six minutes, then filtered through a 40 μm filter. The particle solution is then frozen at −80° C. and lyophilized into dry powder form. The inclusion of 1 mM tazarotene prior to bulk shearing resulted in an average loading of 2±0.4 picomole per particle (FIG. 7).

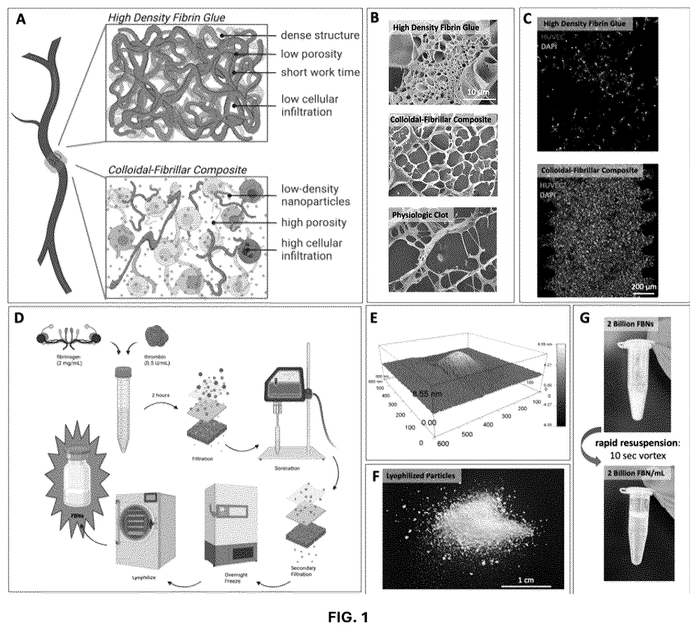

FBN size characterization was done via NanoSight particle tracking analysis (Malvern Panalytical) and atomic force microscopy (AFM, MFP 3D Bio AFM, Asylum). Using 2 mg/mL concentrations of FBNs in deionized water (diH₂O), NanoSight was used to determine concentration of particles per milligram of lyophilized particles from batch to batch. This was used to allow for conversion to particles/mL concentrations of FBNs. NanoSight was also used to determine average hydrodynamic particle diameters. For AFM characterization, FBNs were placed into 0.01 mg/mL solution in sterile PBS. A cleaned glass coverslip was covered in the solution and allowed to dry overnight. Particle characterization was done in air topography mode, with diameter data being collected for 10 particles. When dry, particle diameters were measured at 235.2±39.97 nm (FIG. 1E). In solution, the particles hydrodynamic diameters were measured at 254 nm±75 nm (FIG. 6A).

FBN gels were formed through the addition of CaCl₂ (5 mM final concentration) and FBNs to a patient co-factor. Porcine whole blood was collected through North Carolina State University Tissue Sharing Program. Platelet-poor plasma (New York Blood Center) was used for assays where red blood cells impeded visualization. Human α-thrombin (0.5 U/mL, Enzyme Research Labs) was used to polymerize gels. High-density fibrin glues (HDFG, Tisseel, Baxter; VistaSeal, Ethicon), were used as control surgical sealants throughout these studies. They were stored and formed following manufacturer's instructions, without deviation.

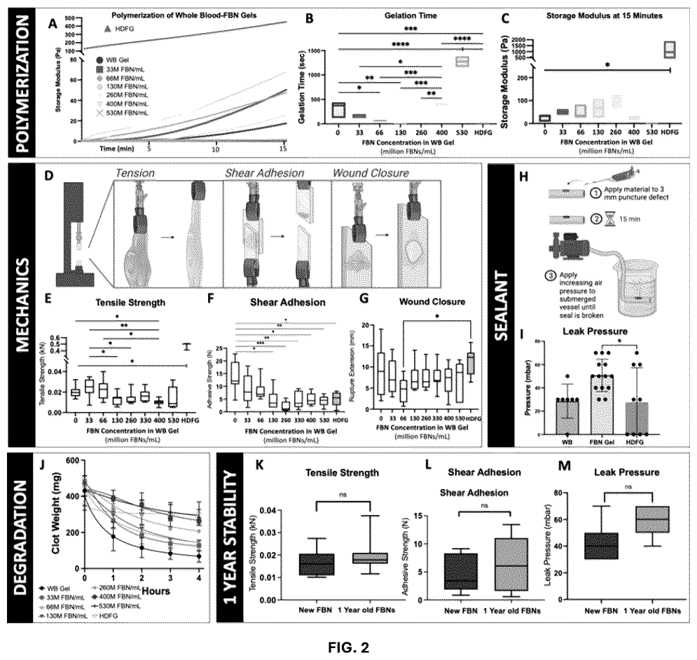

FBN gels were formed through the addition of FBNs to whole blood gel composition. Polymerization of FBN gels was evaluated through rheometric measurement of storage and loss moduli (FIG. 2A). The addition of FBNs to whole blood (WB) resulted in faster gelation time, although this difference was not statistically significant until the addition of 66 million (M) FBN/mL (WB: 310±171 sec, 66M FBN/mL: 66±16 sec; p=0.012; FIG. 2B). The shortest gelation times were reached with the addition of 130M FBN/mL (22±10 sec), after which point increased concentration of FBNs led gelation to increase again. The addition of 260M FBN/mL led to gelation times significantly greater than all lower concentration WB-FBN gels (85±17 sec; p<0.0001). With the addition of 530M FBN/mL, the highest concentration tested, gelation time was significantly greater than that of any other concentration of FBN gel and the whole blood alone (1272±156 sec). High-density fibrin gels polymerized fastest (0 seconds for each sample tested, meaning that polymerization occurred in the 2-5 seconds before the measuring system was able to start recording), although this was not statistically different from the gels formed with 33, 66, 130, or 260 million FBN/mL. Storage modulus after 15 minutes followed a correlative trend, with the addition of increasing concentrations of FBNs to a whole blood gel seeming to increase storage modulus, with peak reached at 260M FBN/mL (101±50 Pa). Beyond that, storage modulus dropped back down (400M FBN/mL: 26±11 Pa, 530M FBN/mL: 1±1 Pa, FIG. 2C), however all of these were trend-level patterns and were not significant. The final storage modulus of high-density fibrin glue (HDFG) was significantly greater than that of any other group and also displayed the greatest variation (956±601 Pa; 0.01<p<0.05 for all comparisons).

To investigate the tunability of FBN glue polymerization dynamics, a rheometer (Anton Paar MCR 92) with a cone-plate fixture was used. For 30 minutes following thrombin-initiated polymerization, oscillating time sweep analysis was performed using 0.5% strain, 1 Hz constant rates at 37° C., measuring every 0.1 seconds. Storage and loss moduli were captured at each measurement. Gelation time was defined as the time at which storage modulus is three times larger than loss modulus. For each group, three replicates were used.

Local maxima in tensile strength were achieved at 33M FBN/mL and 330M FBN/mL (33M FBN/mL: 26±9 N, 330M FBN/mL: 17±6 N). HDFG was significantly stronger than all other groups (HDFG: 474±54 N; 0.01<p<0.05 for all comparisons). The addition of FBNs led to statistically significant decrease in capacity for shear adhesion for all particle concentrations at or above 130M FBN/mL (FIG. 2E). These values, however, were not statistically different from the HDFG samples. Wound closure capacity was largely stable across all groups. HDFG appeared to demonstrate a slightly greater average rupture extension, however this was only significantly different from the 66M FBN/mL gels (66M FBN/mL: 4.6±3.1 N, HDFG: 11.3±3.1 N, p=0.011; FIG. 2F, 8).

To evaluate degradation under the electrolyte balance of the body, 1 mL gel samples were formed and allowed to polymerize for one hour inside individual weight boats, after which they were covered with 0.9% (isotonic) saline. At each hour time point, saline was drained, the gel was blotted, and then weighed. Timing resumed once the gel was re-submerged in saline solution. This proceeded for a total of 4 hours (4 time points). Five replicates were used for each group, except HDFG, for which 3 replicates were used. For 24-hour degradation, gels were left submerged at room temperature for 24 consecutive hours, followed by draining, blotting and weighing only at the end of 24 hours. Here, 3 replicates were used for all groups.

All mechanical assays were performed using an Instron 3400 Series Single-column Universal Testing Machine (Instron, Norwood, MA) and Bluehill software. For tensile studies, 1.5 mL of FBN gel were polymerized overnight, then clamped into the Instron (FIG. 2D). In order to minimize sample pre-load, gels were clamped into the top first and allowed to hang before being clamped below. Tension was administered at a rate of 0.01 mm/s until the point of rupture. Rupture force was recorded for 11-15 replicates per sample group, except for HDFG for which 3 replicates were tested.

As shear strength is most applicable to clinical use for vascular anastomosis, we focused on this aspect of adhesion (FIG. 2D). 50 μL of FBN gel was polymerized for 1 hour between two glass coverslips, each affixed to a glass slide. Slides were clamped in the Instron and extension proceeded at 0.01 mm/sec. Shear strength was recorded for 7-11 replicates per sample group.

To approximate wound closure ability, 0.5 mL of FBN gel was applied to a 0.5 cm linear defect in a 1.5×2.5 cm piece of porcine carotid artery and allowed to polymerize overnight. Adult porcine carotid artery samples were collected from pigs available through the NCSU Tissue Sharing Program. Samples were loaded into the Instron where extension proceeded at a rate of 0.01 mm/s (FIG. 2D, 8). Extension at the point of sealant rupture was recorded for 8-10 replicates per sample type.

For our use of this material as a surgical sealant, it was imperative that we test FBN gels' sealant capacity and burst pressure. To test sealant capacity, 0.5 mL of FBN gel was applied to a 3 mm diameter puncture defect in latex tubing. After allowing 15 minutes for polymerization, the puncture surface was rested on a petri dish of water and 2% aniline blue dye was allowed to flow through. The latex tube was lifted from the surface and any aniline blue dye visible in the petri dish was taken as indication of ineffective seal formation, i.e. a leak pressure of 0 mbar. Tubing samples that passed this leak test were attached to an air pump and submerged in water. With the non-pump side of the tube sealed, increasing air pressure was applied to the tube in 10 mbar increments until biomaterial seal was broken, which was visualized through the appearance of bubbles. Air pressure at time of rupture was recorded as the leak pressure. Between 7 and 15 replicates were used for each sample group.

Sealant capacity was determined by the amount of air pressure required to dislodge a 250 μL sample of biomaterials polymerized in situ as a sealant for a 3 mm puncture wound in latex tubing (FIG. 2G). Based on results from prior mechanical testing, whole blood gels with the addition of 330M FBN/mL were chosen as the ideal candidate for pursuit as a surgical sealant, and for that reason this test focuses on this FBN gel formulation in comparison to HDFG. FBN gel samples outperformed HDFG samples, with significantly higher leak pressure values. (FBN: 51±14 mbar, HDFG: 28±30 mbar, p=0.031; FIG. 2H).

Material degradation was evaluated over the course of 4 hours in isotonic saline solution. Increasing FBN concentration led to decreased degradation over the 4 hours, with 530M and 400M FBN/mL groups performing comparably to HDFG. No quantitative or qualitative evidence of material swelling was noted in either FBN gels or HDFG.

To determine long-term stability of particle properties, we performed head-to-head comparison of 330 million (M) particles/mL FBN gels made with new FBNs (<1 month old, refrigerated) and with FBNs stored at room temperature for 1 year. These comparisons followed the same tensile, adhesive, and leak pressure protocols listed earlier. For tensile testing, 11 replicates were tested per group. For adhesive testing, 9-13 replicates were tested per group. For leak pressure testing, 7 replicates were tested per group.

Tensile strength, shear adhesion, and leak pressure testing procedures were repeated using the FBN gel formulation chosen for pursuit as a surgical sealant (330M FBN/mL) using both newly produced FBNs and FBNs that were stored for 1 year at room temperature in lyophilized powder form. Across all tests, no statistical difference was noted between the new and old FBN groups (FIG. 2K, 2L, 2M). This demonstrates the long-term room temperature stability of lyophilized FBNs.

FBN gel structure was imaged and characterized using both confocal microscopy and cryogenic scanning electron microscopy (cryoSEM), allowing for both gross and detailed visualization. For confocal microscopy, 50 μL FBN gels with 2.5% (w/w) Alexa-Fluor 488 labeled fibrinogen (Thermo Fisher Scientific) were polymerized between a glass slide and coverslip for 1 hour, then imaged using a Zeiss Laser Scanning Microscope (LSM 710, Zeiss Inc.) at 40× magnification.

Five clots were formed per gel type, and a minimum of 3 random 5.67 μm z-stacks were acquired for each gel. ImageJ (National Institutes of Health) was used to produce 8-bit z-stack projections. Gel fiber density was determined by thresholding the images to produce a black and white binary image and then calculating the ration of fiber to background pixels in each image. For cryoSEM imaging, FBN gels of 150 μL were formed in 0.6 mL Eppendorf tubes and allowed to polymerize for 12 hours. Clots were rapidly plunge-frozen in super-cooled liquid nitrogen, fractured with a cold knife, etched for 10 minutes, and sputter-coated with gold. Imaging was performed at 1000×, 1500×, 2000×, and 2500× on two samples per gel type, in at least three random locations within the clot. ImageJ (National Institutes of Health) was used to perform image segmentation and determine pore size and percent porosity. MatLab was used to analyze fiber alignment.

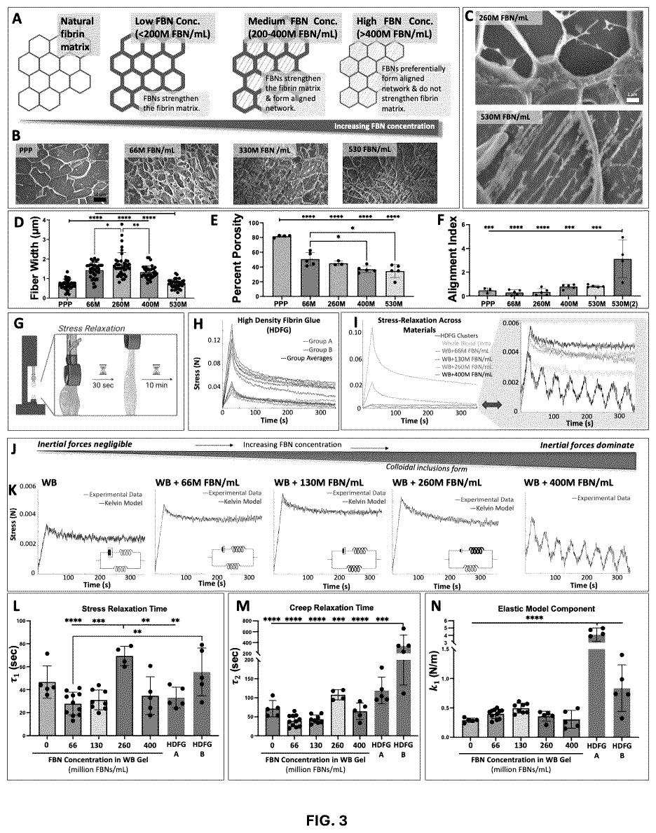

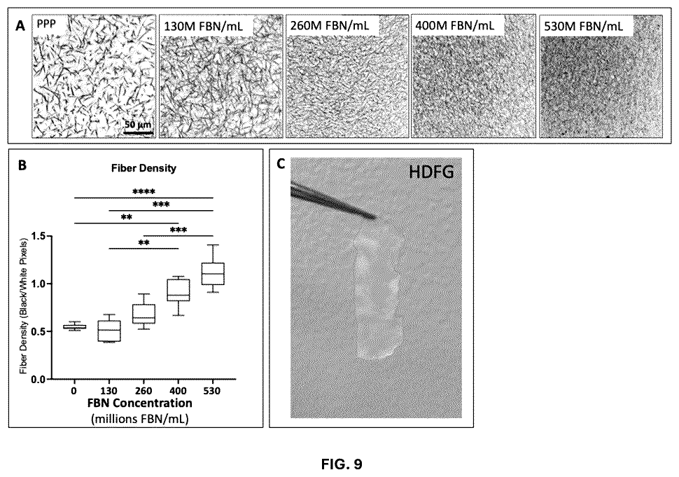

Preliminary structural analysis of FBN gels with different particle concentrations using confocal microscopy demonstrated increased density as FBN concentration within the gel increased (FIG. 9A, 9B). These images, however, became indistinct with increased particle concentration, which led to the pursuit of more detailed imaging modalities. Through cryogenic scanning electron microscopy (cryoSEM) of FBN gels, qualitative patterns in FBN gel scaffold structure became apparent (FIG. 3A). At lower concentrations of FBNs (<200M FBN/mL), FBNs are primarily found within the natural fibrin matrix resulting in a structurally similar, but more robust fibrin network (FIG. 3A, 3B, 3C). At higher concentrations (>400M FBN/mL), FBNs are primarily found outside of the natural fibrin matrix forming a highly aligned secondary structure, and the “natural” fibrin network does not appear strengthened (FIG. 3A, 3B, 3C). Between these two extremes, in the intermediate particle concentrations (^{˜}200-400M FBN/mL), we see aspects of both structural regimes: strengthened fibrin matrix, as well as what appears to be intrapolymerization among FBNs to form a thin secondary highly-aligned fibrin scaffold. Quantitative analysis of cryoSEM images validates these conclusions, demonstrating that the addition of FBNs increases average fiber thickness, reaching a peak at 260M FBN/mL (1.7±0.6 μm), and then tapering back down to approximately baseline at 530M FBN/mL (PPP: 0.71±0.28 μm, 530M FBN/mL: 0.73±0.23 μm; FIG. 3D). As particle concentration increases, porosity decreases incrementally, with significant difference from baseline across all FBN gel concentrations (PPP: 81±2%, 66M FBN/mL: 51±9%, 260M FBN/mL: 45±3%, 400M FBN/mL: 37±5%, 530M FBN/mL: 35±9%, p<0.0001 for all comparisons with PPP; FIG. 3E). Finally, increasing particle concentration leads to no change in alignment of the primary structure, but analysis isolating the secondary structure at 530M FBN/mL—labeled in FIG. 3F as 530M(2)—showed that this secondary structure demonstrated significantly greater alignment index (AI) than any FBN gel's primary alignment (530M(2): 3.1±1.6 Al, PPP: 0.46±0.22 Al, 66M: 0.29.0.23 Al, 260M: 0.34±0.29 Al, 400M: 0.79±0.20 Al, 530M: 0.82±0.11 Al, p<0.0003 for all comparisons with 530M(2); FIG. 3F).

As it is well-established that blood clots display viscoelastic material properties, we pursued a standard linear viscoelastic (Kelvin) model to describe the stress-relaxation properties of our whole blood FBN (WB-FBN) gels. In this model, which can be thought of as a mechanical circuit, the time-dependent viscosity component of the material is modeled by a dashpot and a spring in series (spring constant k₂>0, damping coefficient c>0, respectively). A second spring in parallel with the first component models the elastic properties of the material (spring constant k₁>0). This model may be expressed in a simple mathematical form:

$$F ( t ) + {τ}_{1} \cdot {F}^{′} ( t ) = {k}_{1} [ u ( t ) + {τ}_{2} \cdot {u}^{′} ( t ) ] ,$$

Where:

$${τ}_{1} = \frac{c}{{k}_{2}} , {τ}_{2} = c [ \frac{1}{{k}_{1}} + \frac{1}{{k}_{2}} ] , and {τ}_{2} > {τ}_{1}$$

Here F(t) represents force as a function of time acting on the viscoelastic solid and u(t) represents displacement (stretch) over time. In this expression, T₁ may be thought of as the stress-relaxation time and T₂ may be thought of as the creep-relaxation time. We chose to use the Kelvin model, as it is the simplest model that accounts for both stress relaxation and creep. There are many extensions of the Kelvin model, through the addition of further parallel components, but our goal here was to determine and apply the simplest model that was able to reflect the viscoelastic properties of the WB-FBN gels.

We solved the Kelvin model as it relates to a ramp and hold displacement with a ramp time tₚ, final displacement uₚ, and initial force on the sample is represented by Fₒ:

$$For t < {t}_{p} : F ( t ) = \frac{{k}_{1} \cdot {τ}_{2} \cdot {u}_{p}}{{τ}_{1}} \cdot \frac{t}{{t}_{p}} + {F}_{o} \cdot {e}^{- \frac{t}{{τ}_{1}}} - {k}_{1} \cdot \frac{{τ}_{2} - {τ}_{1}}{{τ}_{1}^{2}} \cdot \frac{{u}_{p}}{{t}_{p}} \cdot ( {τ}_{1} \cdot t - {τ}_{1}^{2} + {τ}_{1}^{2} \cdot {e}^{- \frac{t}{{τ}_{1}}} )$$

$$For t \geq {t}_{p} : F ( t ) = \frac{{k}_{1} \cdot {τ}_{2} \cdot {u}_{p}}{{τ}_{1}} \cdot \frac{t}{{t}_{p}} + {k}_{1} \cdot {τ}_{2} ( \frac{{u}_{p} - {u}_{p} \cdot \frac{t}{{t}_{p}}}{{τ}_{1}} ) + {F}_{o} \cdot {e}^{- \frac{t}{{τ}_{1}}} - {k}_{1} \cdot \frac{{τ}_{2} - {τ}_{1}}{{τ}_{1}^{2}} \cdot [ \frac{{u}_{p}}{{t}_{p}} ( {τ}_{1} \cdot t - {τ}_{1}^{2} + {τ}_{1}^{2} \cdot {e}^{- \frac{t}{{τ}_{1}}} ) + {τ}_{1} ( {u}_{p} - {u}_{p} \cdot \frac{t}{{t}_{p}} ) + \frac{{u}_{p} \cdot {τ}_{1}^{2}}{{t}_{p}} - \frac{{u}_{p}}{{t}_{p}} \cdot {τ}_{1}^{2} \cdot {e}^{\frac{{t}_{p} - t}{{τ}_{1}}}$$

For the acquisition of stress-relaxation curves for validation of the Kelvin model, 0.5 mL WB gels, WB-FBN gels, and HDFG gels were produced using earlier described methods. Gels were formed into roughly 2 cm by 1 cm rectangles in weigh boat and allowed to polymerize overnight. Upon being clamped into the Instron UTS, gels were stretched (1 mm/sec) for 30 seconds, followed by 10 minutes held without additional displacement. Force measurements were taken every 0.1 seconds throughout stress and relaxation. Resultant curves were cleaned of noise by using a 20 point (2 second) moving average. The presence of outlier curves was determined through co-localized plotting and removal of grossly variant curves.

An initial estimate for t₂, and k₁, was made based on an initial guess for τ₁ and calculated values derived from observed experimental values—time at peak (tₚ), final displacement (uₚ), initial force (Fₒ), peak force (Fₚ), and equilibrium force (F_{∞}):

$${k}_{1} = \frac{{F}_{∞}}{{u}_{p}}$$

$${τ}_{2} = {τ}_{1} + \frac{1}{1 - {e}^{- \frac{{t}_{p}}{{τ}_{1}}}} \cdot \frac{{t}_{p}}{{F}_{∞}} \cdot ( {F}_{p} - {F}_{∞} - {F}_{o} \cdot {e}^{\frac{{t}_{p}}{{τ}_{1}}} )$$

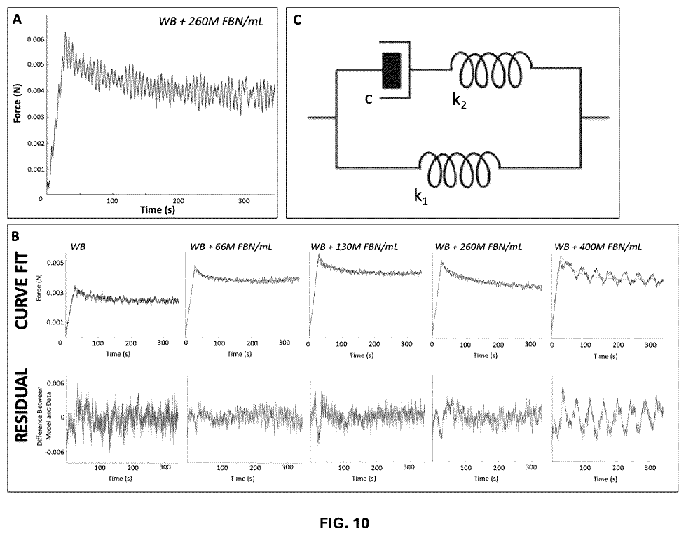

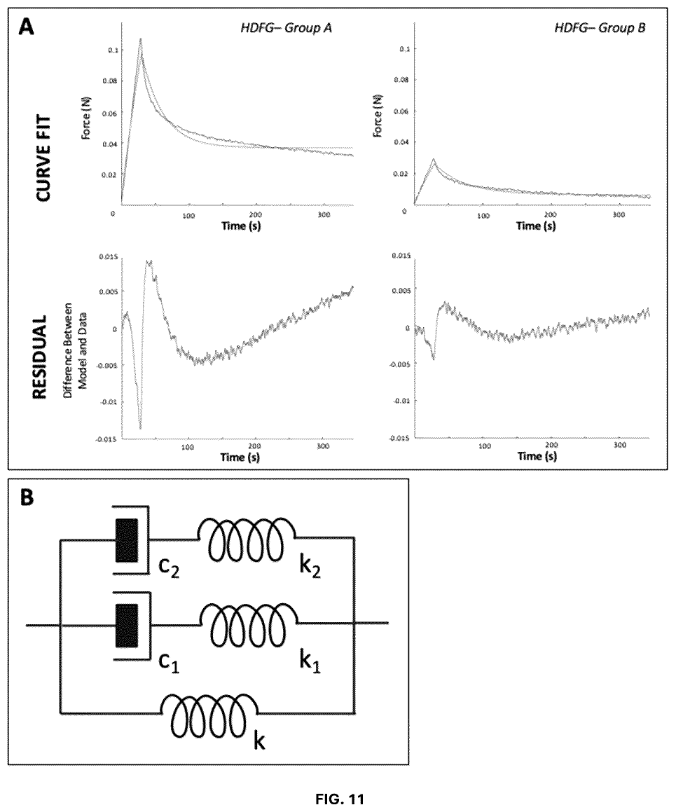

This first estimation was used to initialize a least squares optimization via MatLab using a Nelder-Mead simplex (direct search) method (“fminsearch”). This resulted in estimated values for. T₁, T₂, and k₁, which were found to be robust to perturbation of the initial guess. Cost and residuals were evaluated for goodness of fit (FIG. 10), resulting in validation of the use of a Kelvin model for WB gels and WB-FBN gel groups with the addition of <400M FBN/mL. Curves from WB-FBN gels with 400M FBN/mL clearly demonstrated wave-like properties indicative of a regime in which inertial forces dominate. A Kelvin model adequately approximated curves from HDFG samples, but was not ideal (FIG. 11). An extended Kelvin model, with the addition of another parallel kelvin body component (dashpot and spring in series), was applied to all samples, but only made a meaningful improvement in cost for HDFG samples. A Kelvin model was used to compare parameters across gel types.

Tensile stress-relaxation curves for FBN gels of varying concentrations, whole blood gel, and HDFG were gathered through the use of an Instron UTS (FIG. 3G). The HDFG data from these experiments were more heterogeneous than any other group, with an even split between two stress-relaxation curve patterns (FIG. 3H). This structural heterogeneity aligns with the structural heterogeneity notable in the leak pressure data analysis and visible in images of polymerized gels (FIG. 2I, 9C). It appears that the thrombin initiates polymerization too quickly to ensure proper mixing. Stress-relaxation curves varied visibly between gel types, but only the HDFG curves were clearly divided into subgroups (FIG. 3I). Whole blood gels and FBN gels with lower particle concentrations predominantly acted within a viscoelastic solid regime. With the addition of greater colloidal inclusions, FBN gels demonstrated greater inertial noise, until inertial forces dominated the stress-relaxation curve at 530M FBN/mL particle concentrations, made evident by the clear wave-type regime of the relaxation phase at this concentration (FIG. 3J, 3K, S5A, 10B). Differences between stress-relaxation curves were quantified by fitting them with a Kelvin model to determine characteristic material parameters (FIG. 3K, 10C). Residuals confirmed that this model was an appropriate choice for application to FBN gels until the regime with inertial effects was reached in the FBN gels with 400 million FBNs/mL (FIG. 10). While the fit was less appropriate for HDFG, which was more appropriately fit by an extended Kelvin model, to allow for comparison between gel types, it was important to compare them using the same model (FIG. 11). The 260M FBN/mL gels had significantly greater stress-relaxation times than any of the other FBN gels and the first of the two HDFG subpopulations (69±9 sec; p<0.01 for all comparisons; FIG. 3L). Differences in strain relaxation time were insignificant, aside from that of the second HDFG subpopulation, which was significantly different from all other groups (p<0.001 for all comparisons; FIG. 3M). As displacement was the independent variable in curves predicted using the model, it follows that the comparison of strain relaxation time was not as meaningful. With the addition of FBNs to the whole blood gel, the elastic model component of the curves increased through 130M FBN/mL gels, and then decreased again through 530M FBN/mL gels, however these trends were not statistically significant. Both HDFG subpopulations had a greater elastic model component than all other groups, with HDFG-A being significantly higher than all other group, including HDFG-B (p<0.0001 for all comparisons; FIG. 3N).

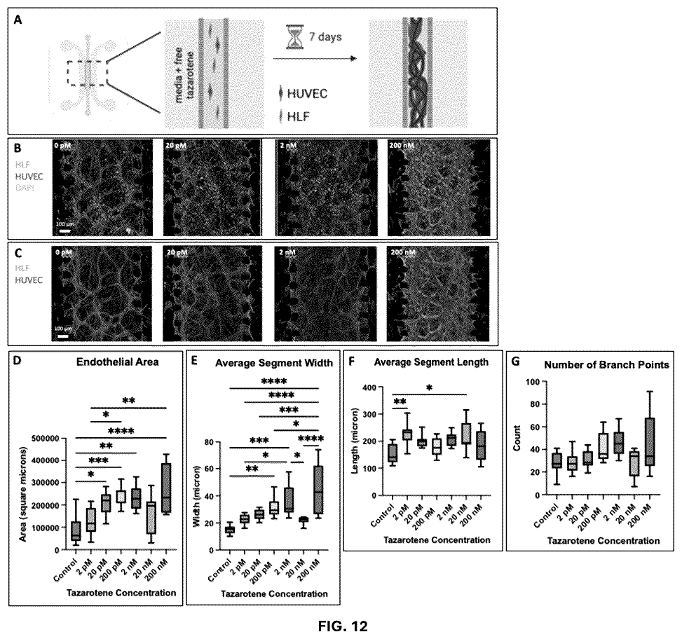

The effects of tazarotene dosage on vascular network formation were investigated through the use of an established microfluidic vasculogenesis model (FIG. 12A). HUVECs and HLFs were embedded in 2 mg/mL human fibrin gels made 25 mM HEPES buffer and 0.5 U/mL thrombin. Media channels in the microdevice (FIG. 12A) were filled with HUVEC media supplemented with free tazarotene. Daily media changes and brightfield imaging were completed until cells were fixed with 4% paraformaldehyde at 1 week after seeding. Cells were permeabilized with 0.3% Triton X-100 (Sigma), immunostained with PEKAM-1 antibody (ThermoFisher), and stained with phalloidin (Invitrogen) and DAPI (Invitrogen) in order to visualize networks of both HUVECs and HLFs. Imaging was performed using a confocal microscope with a 10× objective. At least 3 devices were imaged for each experimental group and 3 images were taken per device. ImageJ was used to quantify vascular network characteristics (average segment length, average segment width, average number of branch points, and total endothelial area).

For analysis of vascular network response to the biomaterials used in our in vivo surgical studies, we used a permutation of this vasculogenesis assay. Instead of embedding cells in fibrin gels in the central channel, cells were embedded in FBN gel (330 M FBNs/mL), tFBN gel, or HDFG, and cells were cultured with regular HUVEC media. Cell culture and imaging proceeded as described earlier. At least 3 devices per biomaterial were cultured with HUVECs and HLFs and at least another 3 devices were only cultured with HUVECs.

The effects of tazarotene released from bulk fibrin gels on individual cellular morphology was determined through the low-density culture of HUVECs on thin films of loaded fibrin gels. Fibrin gels with varying concentrations of bulk tazarotene incorporated were allowed to polymerize between two clean glass coverslips for 1 hour, in order to form thin films. Thin films of 80 μL were made using 2 mg/ml human fibrinogen (Enzyme Research Laboratories), HEPES buffer (25 mM), and 0.5 U/ml human thrombin. Upon removal of one glass coverslip, the remaining glass and attached gel were placed within the well of a 12-well plate and rinsed with PBS. HUVECs (15,000 cells/well) were cultured for 16 hours, at which point they were fixed and stained. Images of cells were captured at 10× using an EVOS FL Auto Imaging System, with at least 5 images taken per thin film. In order to validate the nanoparticle release of tazarotene from FBNs, 80 μL FBN gel (330 M particles/mL) thin films were formed with different relative ratios of tazarotene-loaded to unloaded FBNs. All other elements of the procedure were the same.

At this point, we selected our optimized formulations for sealant functionality. Based on our mechanical and structural data, we decided that a concentration of 330 million FBN/mL would optimize mechanical capacity while allowing us to gain the potential healing benefits of the secondary aligned structure that appears above concentrations of 200 million FBN/mL. However, we wanted to take advantage of the localized nanoparticle drug delivery capacity afforded by the use of FBNs. Prior work describing the positive effects of tazarotene on vascular endothelial cell migration and angiogenesis showed promise for its application to gross vascular healing. Thus, it was important for us to investigate the potential effects of tazarotene on vascular network formation and determine an optimal tazarotene dosage for nanoparticle delivery. To this end, we employed a vasculogenesis assay wherein human umbilical vein endothelial cells (HUVECs) and human lung fibroblasts (HLFs) were embedded in fibrin gel within a microfluidic device and co-cultured for one week with exposure to different concentrations of free tazarotene in the media (FIG. 12A). Visually, the resultant HUVEC networks become larger and individual segments become wider through 2 nM tazarotene concentration, beyond which the network remains dense but also becomes more disorganized (FIG. 12B, 12C). Quantification of the HUVEC network morphology confirmed that all tazarotene concentrations tested between 20 and 2000 μM resulted in significantly greater endothelial network area compared to control (control: 89,852±64,861 μm², 20 μM: 206,763±51,803 μm², 200 μM: 240,770±44,210 μm², 2000 μM: 230,087±51,803 μm², control vs 20 μM: p=0.015, control vs 200 μM: p=0.0006, control vs 2000 μM: p=0.0018; FIG. 12D) and exposure to 200 μM tazarotene resulted in significantly greater endothelial network area compared to 2 μM (2 μM: 130167±61886 μm², p=0.0257). Increased free tazarotene concentration resulted in increased average network segment width, with the values for both 200 μM and 2000 μM groups being significantly greater than control (control: 15±3 μm, 200 μM: 31±7 μm, 2000 μM: 36±11 μm, control vs 200 μM: p=0.0077, control vs 2000 μM: p=0.0002; FIG. 12E). The average segment width at 2000 μM was also significantly greater than the 2 μM group (2 μM: 23±4 μm, p=0.046). Average segment length was significantly greater than control in the 2 μM and the 20 nM groups (control: 154±36 μm, 2 μM: 226±40 μm 20 nM: 218±54 μm; control vs 2 μM: p=0.0063, control vs 20 nM: p=0.022, FIG. 12F). No significant differences in branch points were found between groups (FIG. 12G).

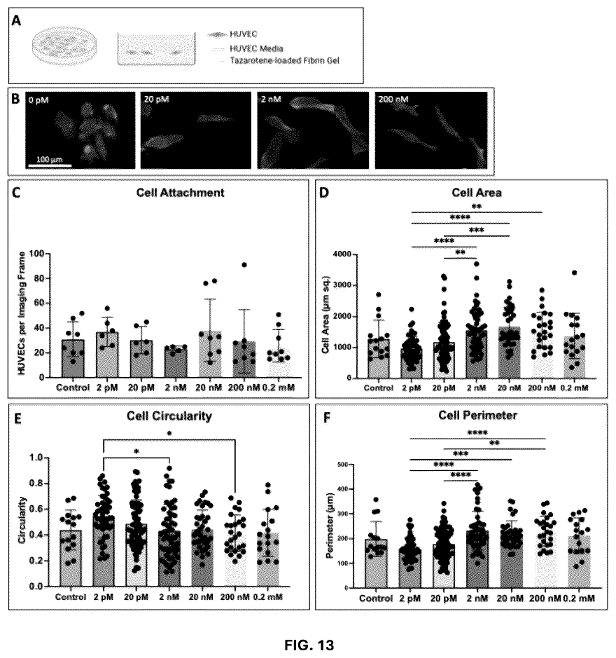

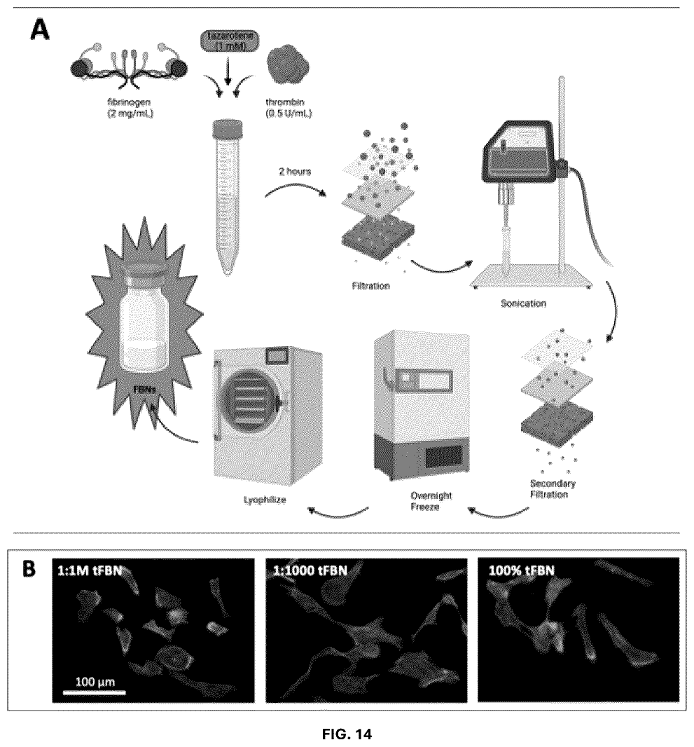

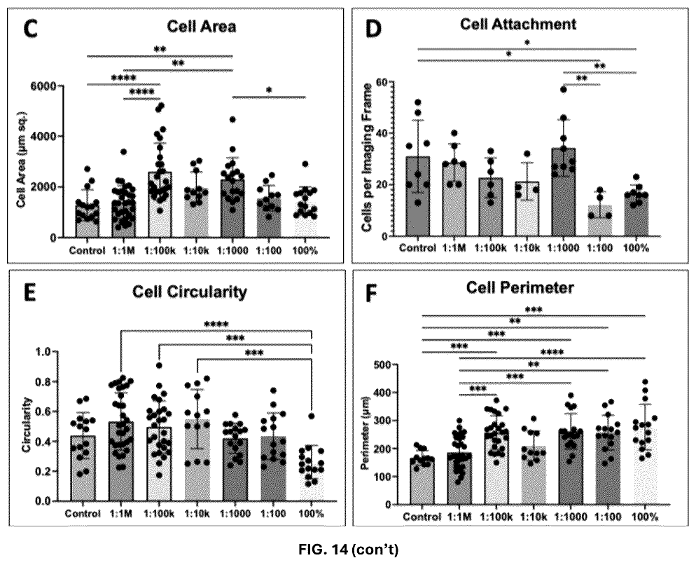

To evaluate effects of tazarotene on individual cellular morphology, HUVECs were cultured on thin films of fibrin gel that were bulk loaded with different concentrations of free tazarotene (FIG. 13A, 13B). Different concentrations of tazarotene had no significant effect on cellular attachment (FIG. 13C). With increasing concentrations of tazarotene, more cell spreading was observed, with the most cell spreading observed at 200 nM, after which cells spread less (FIG. 13D). With increasing concentrations, cells also exhibited less circularity and greater perimeter (FIG. 13E, 13F). Altogether, this data is indicative of a relatively wide tazarotene dosage band which appears to promote HUVEC activation. To validate the delivery of tazarotene by FBNs, HUVECs were cultured on thin films of FBN gel that were made with 330M FBN/mL, but differing ratios of unloaded to tazarotene-loaded FBNs to produce different effective doses of tazarotene. The resultant data validated similar trends to those using free tazarotene, allowing for reasonable expectation that tazarotene-loaded FBNs do indeed release tazarotene when incorporated into FBN gels and the resultant cellular effects are not altered by nanoparticle delivery (FIG. 14).

For analysis of HUVEC cellular morphology response to the biomaterials used in our in vivo surgical studies, we used a variation of the thin film protocols already described. Cells were cultured in the same manner on thin films of FBN gel, tFBN gel, and HDFG. Staining, imaging, and quantification proceeded following the earlier description. At least 3 thin films were analyzed per sample group, with at least 5 images taken per thin film.

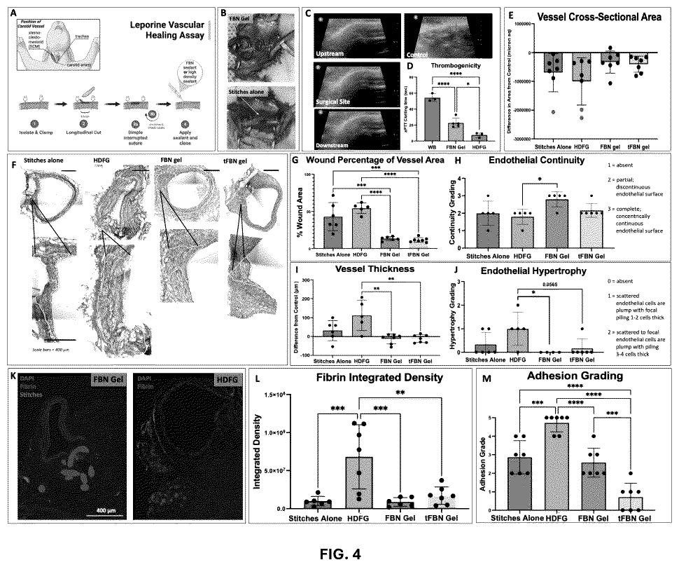

All in vivo experiments were approved by North Carolina State University Institutional Animal Care and Use Committee and conducted in Association for Assessment and Accreditation of Laboratory Animal Care (AAALAC) international-accredited facilities. Our studies used 90 to 150-day old New Zealand White rabbits (Charles River Laboratories) in order to investigate vascular healing following the use of FBN and tFBN gels as an adjunct sealant material following suture repair of a carotid artery injury (FIG. 4A). While distinct from previous studies, our in vivo model follows established models for assessing vascular healing. Rabbits were pre-treated for pain management with hydromorphone (0.1-0.3 mg/kg) or buprenorphine (0.05 mg/kg). Prior to anesthesia, rabbits were sedated with a one-time intramuscular injection of ketamine (10-20 mg/kg) and dexmedetomidine (0.025 mg/kg). Upon achievement of appropriate sedation, rabbits were intubated and anesthesia and analgesia persisted throughout surgery with maintenance via isoflurane (<5%), as well as ketamine constant rate infusion (CRI, 0.2-1.2 mg/kg/hr), lidocaine CRI (50-100 mcg/kg/hr), buprenorphine (0.05 mg/kg IV) and dexmedetomidine boluses during surgery (0.025-0.25 mg/Kg IM). A bupivacaine incisional block (2 mg/kg) was administered locally prior to incision into the neck and following final closure. Vital signs were monitored throughout surgery by dedicated staff.

Rabbits were fasted for 1-2 hours prior to surgery to minimize the likelihood of complications during intubation. Following sedation and intubation, the rabbit was placed in dorsal recumbent position and the neck was shaved and cleaned with alternating antiseptic and alcohol scrubs. The right carotid artery was isolated from the surrounding connective tissue and muscle by using metal ring clips. The right carotid artery (CA) of each rabbit was clamped, longitudinally cut between the clamps, then repaired with 6-0 PDS using interrupted sutures with <1 mm between each to reduce the likelihood of stenosis or hypertrophy. Suture-line was sealed with 0.2 mL FBN gel, tFBN gel, HDFG, or nothing, with 7 rabbits per treatment group. 3 mL of blood was drawn from each animal after sutures were placed, which was used as a patient cofactor to make the FBN gel or tFBN gel in those treatment groups. The closure was performed in a layered fashion: muscle layer, fascia, subdermal, and dermal layers each being closed sequentially. The closure used dissolvable sutures. Antisedan and atipamezole were administered as needed as reversal agents due to prolonged anesthesia. Prior to recovery from anesthesia, ultrasound was used to validate the patency of the surgical vessel and compare it to the non-surgical side.

Ultrasound imaging (Butterfly iQ) was performed at 1 week and 2 weeks after surgery to evaluate vessel patency. Prior to imaging, rabbits were sedated with a one-time intramuscular injection of ketamine (10-20 mg/kg) and dexmedetomidine (0.025 mg/kg). At 3 weeks after surgery, carotid arteries and surrounding tissues were collected in a 10% sucrose PBS solution. Tissues were exposed to a serial dilution (10%, 20%, 30% sucrose, each for 24 hours), prior to embedding in optimal cutting temperature (OCT) medium. A 28-gauge syringe was used to fill the vessel lumen with OCT to minimize vessel tissue damage during freezing. Embedded tissues were frozen gradually via exposure to dry ice. After hardening of the OCT, embedded tissues were stored at −80° C.

Sectioning of tissues proceeded using a cryotome set to 5 μm thickness. Sections were fixed in 4% paraformaldehyde for 15 minutes. Hematoxylin and eosin (H&E) staining were performed using established protocols. These H&E sections were imaged using the 10× objective of an EVOS FL Auto Imaging System, with at least 3 sections imaged per animal. These images were analyzed for vessel cross-sectional area, wound percentage of vessel, and vessel thickness using ImageJ. H&E sections were also analyzed by a veterinary pathologist for inflammation grading.

Fibrin immunohistochemistry was used to identify both any remnant FBNs or HDFG, as well as any evidence of widespread fibrin deposition. Anti-fibrin antibody (UC45) (Genetex 19079, 1:67 dilution), was used for this imaging, alongside a goat-anti-mouse secondary antibody. All samples were counter-stained using VectaShield mounting medium with DAPI. Samples were imaged using the 10× objective of an EVOS FL Auto Imaging System, with at least 3 sections imaged per animal. These images were analyzed for fibrin integrated density using ImageJ.

Prior to transcriptomic analysis, RNA extraction was performed on remaining cryo-preserved vessel tissue. Frozen tissue was homogenized in 0.5 mL of lysis buffer (Invitrogen) with the use of a beadmill. RNA extraction proceeded through the use of a TRlzol Plus RNA Purification Kit (Invitrogen), following the manufacturer's instructions without deviation. Total purified RNA concentrations were determined using a NanoDrop 1000 spectrophotometer (Thermo Scientific). NanoDrop was also used to verify that all RNA samples were of appropriate purity (260/230 ratio>1.8) for transcriptomic analysis. For quantification of RNA expression by tissues, we used the purified RNA samples in a NanoString nCounter study using a custom codeset (selected group of genes). NanoString protocols for sample preparation and testing were followed without deviation.

Biomaterial thrombogenicity was compared using a modification of established techniques for determining activated partial thromboplastin time (aPTT) testing. A whole blood aPTT test was performed following manufacturer's instructions, with the addition of 20 μL of lyophilized biomaterial at the bottom of the testing well. Time for gelation to occur was recorded for 3-5 replicates per sample group.

At this point, we selected our optimized formulations for appropriate localized drug delivery. For our tazarotene-loaded FBN gels (tFBN gels), we decided to use a relative ratio of loaded to unloaded FBNs ( 1/1000) that gave us the best combination of endothelial cell attachment and activation. To further investigate the wound healing properties of our selected formulations of FBN and tFBN gels, we applied these materials to a common clinical problem that benefits from the use of a surgical sealant: leak prevention in vascular repair. In these studies, FBN gel, tFBN gel, or HDFG was used as a sealant around successful closure with simple interrupted sutures (FIG. 4A, 4B). Treatment with sutures alone, without an adjunct sealant material, was used as a control group. Ultrasound was used to evaluate vessel patency immediately following surgery, as well as at weekly intervals following surgery until the time of euthanasia (FIG. 4C). Treatment course for all surgically-treated rabbits was uncomplicated, with no notable adverse events. Two rabbits experienced cardiac arrest in response to initial sedation and intubation prior to surgery, however this outcome is not uncommon for prey animals. Only three rabbits out of 28 experienced complete stenosis of the injured vessel. In all three cases, ultrasound immediately following surgery demonstrated patency, and stenosis was noted in the ultrasound taken one week after the initial surgeries. Two of three occurred in the HDFG group, and the third occurred in the control group. HDFG material properties may have increased the likelihood of thrombosis leading to stenosis, as HDFG was found to be significantly more thrombogenic than FBN gel in an assay where whole blood was exposed to fully polymerized biomaterial and tested for aPTT clotting time (p<0.0001, FIG. 4D). Even with the inclusion of these stenosed rabbits, vessel cross-sectional area was not significantly different between any of the treatment groups (FIG. 4E). From stained vessel cross sections, it was apparent both visually and through quantification that FBN gels resulted in significantly smaller wounds as a percentage of total vessel cross-sectional area than either stitches alone or HDFG (stitches alone: 43±19%, HDFG: 54±7%, FBN gel: 13±3%, stitches alone vs FBN: p=0.0004, HDFG vs FBN gel: p<0.0001; FIG. 4F, 4G). This was also true of tFBN gels (tFBN gel: 11±3%, stitches alone vs tFBN: p=0.0001, HDFG vs tFBN: p<0.0001). This analysis did not include stenosed vessels.

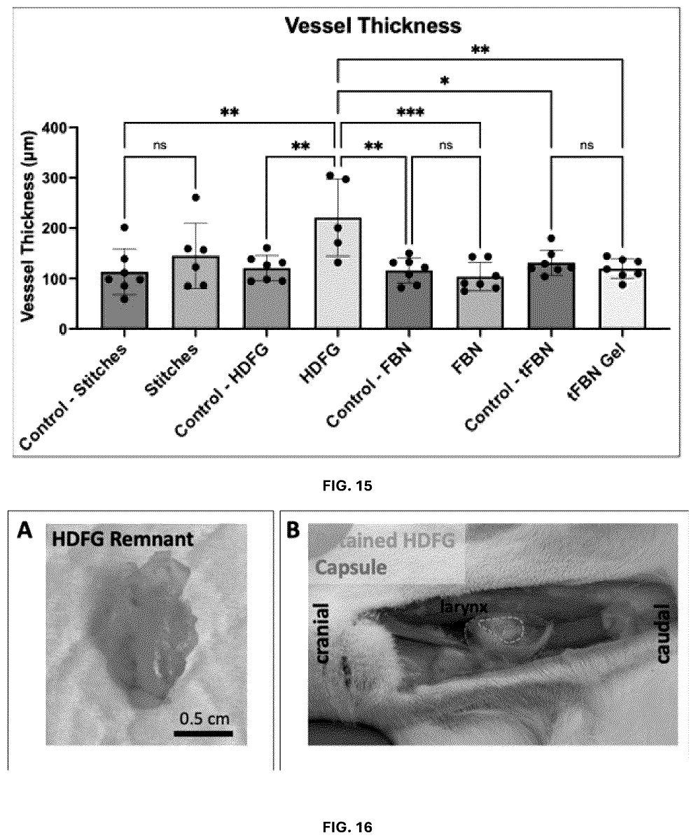

A three-week endpoint for this study was chosen in order to allow for evaluation of the potential formation of neointimal hyperplasia at the wound site after surgery. This negative outcome is not uncommon in vascular surgery and increases risk for vessel (re)stenosis. As such, it is an important consideration in evaluation of a new wound healing material for vascular applications. To this end, vessel thickness was quantified, making it clear that among animals treated with either FBN or tFBN gel, there was no significant difference between the surgical and non-surgical vessel thickness (FIG. 15). HDFG-treated vessels' difference from baseline were significantly larger than those of either FBN gels or tFBN gels, and both FBN and tFBN gels resulted in slightly thinner arterial walls than at baseline, although this difference was not significant (HDFG: 112±81 μm, FBN: −12±26 μm, tFBN: −12±21 μm, HDFG vs FBN: p=0.001, HDFG vs tFBN: p=0.001; FIG. 4H, 15) These results indicate preliminary evidence that neointimal hyperplasia is not a significant risk with these FBN gels and tFBN gels, although longer timescales should be investigated in future studies.

At 3 weeks after surgery, excessive inflammation is a negative surgical outcome. Inflammation was quantified via pathological characterization scale.

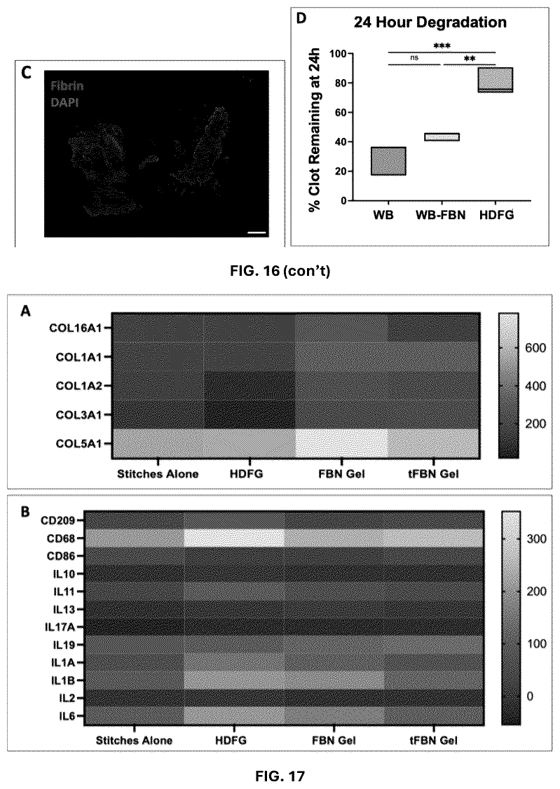

Fibrin immunohistochemistry (IHC) allowed for the visualization of both FBN remnants and HDFG remnants at 3 weeks post-operation (FIG. 4J). Differences in both fibrin material remnant volume and diffuse fibrin deposition at the wound site were captured through comparison of fibrin integrated density of fibrin IHC images. Fibrin at the wound site in the HDFG-treated group was significantly higher than all other groups (FIG. 4K). This is of clinical concern, as fibrin deposition has been identified as a risk factor for surgical adhesion formation. Additionally, and not included in this quantification, significant retained pieces of HDFG with surrounding capsule formation were found at the wound site during tissue collection (FIG. 16A). Fibrin immunohistochemistry (IHC) confirmed that the material was retained fibrin glue (FIG. 16B), and this lack of degradation was corroborated by in vitro degradation comparison (FIG. 16C). Surgical adhesions were also graded on a 5-point scale immediately following vessel tissue collection. HDFG-treated animals had significantly greater extent of surgical adhesion to the vessel wound site than any other treatment group (HDFG vs stitches alone: p=0.0006, p<0.0001 for all other comparisons, FIG. 4L). While FBN gels had no significant difference in surgical adhesions when compared to stitches alone, tFBN gels exhibited significantly lesser extent of surgical adhesion than either stitches alone or FBN gel (stitches alone vs tFBN: p<0.0001, FBN vs tFBN: p=0.0006, FIG. 4L).

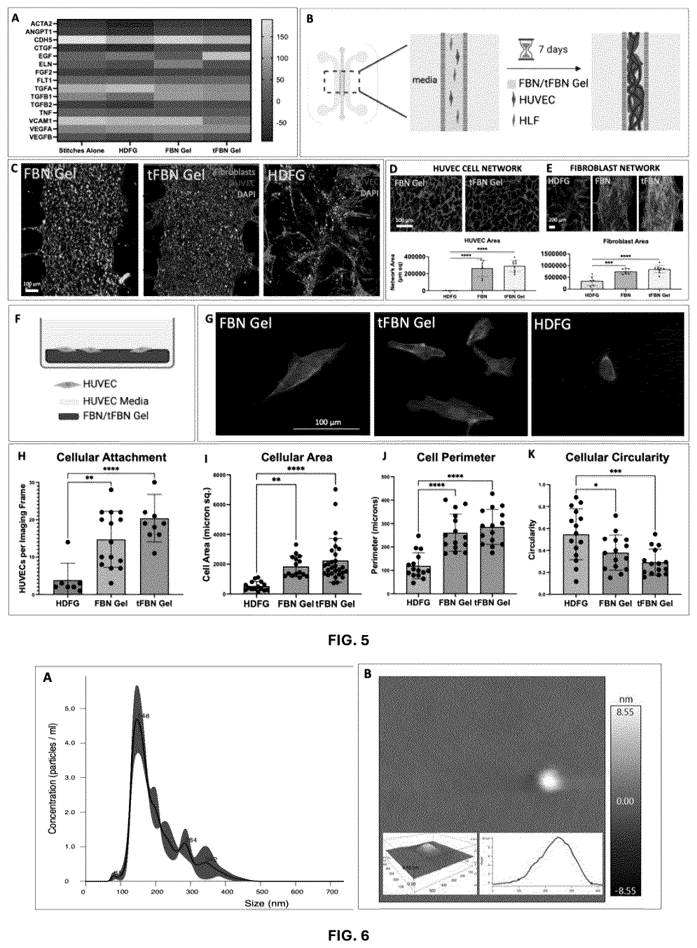

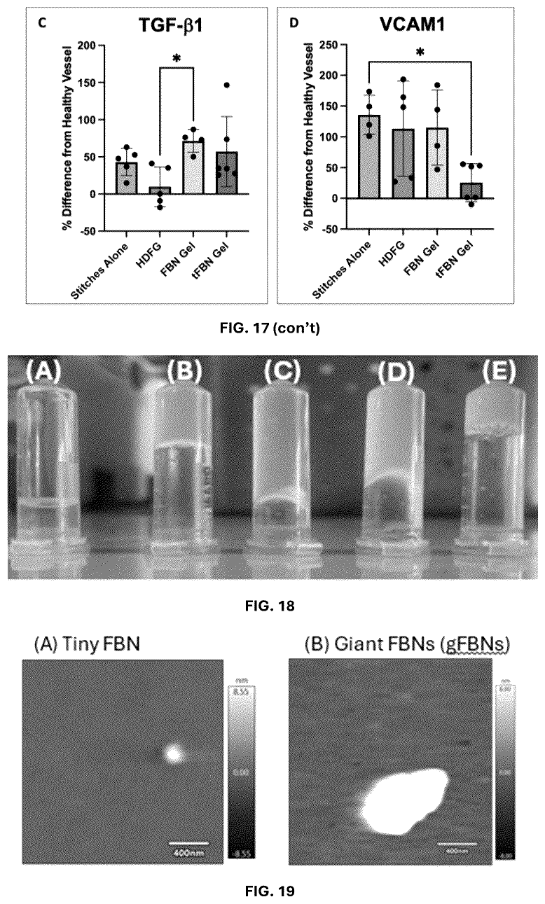

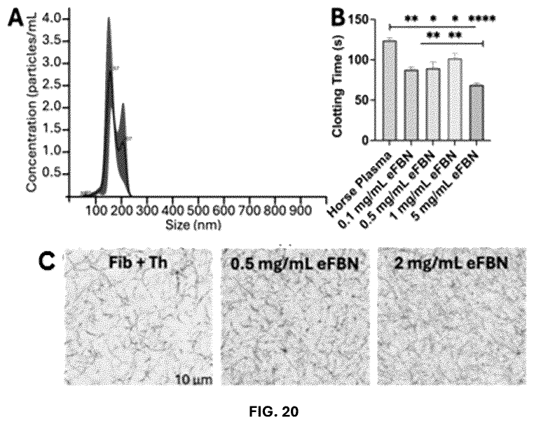

We pursued transcriptomic analysis of wound site tissue to attempt to tease out potential mechanistic causes of the differences seen in surgical healing outcomes in vivo. Trends emerged, suggesting less collagen presence and greater inflammatory markers in the HDFG-treated tissue, although these differences were not statistically significant (FIG. 17A, 17B). FBN gel-treated tissue expressed significantly more TGF-β1 than HDFG-treated tissue (HDFG: 10±27 percent difference from healthy vessel tissue, FBN: 72±15% difference from healthy vessel tissue, p=0.047, FIG. 5A, 17C). The only notable expressive difference observed between FBN gel-treated tissue and tFBN gel-treated tissue, was in the expression of VCAM1, which tFBN-treated tissues expressed less of (FIG. 17D). All surgically-treated tissues expressed significantly more VCAM1 than control (non-surgical) vascular tissue, except tFBN-treated tissues. Vascular tissues treated with tFBNs also expressed significantly less VCAM1 than the stitches alone group (Stitches Alone: 136±32% difference from healthy vessel tissue, tFBN: 26±31% difference from healthy vessel tissue, p=0.028, FIG. 5A, 17D).

To evaluate differences in cellular effects of the materials tested in vivo, we utilized both network-level and cell-level analysis. By using an established vasculogenesis assay wherein HUVECs and HLFs were embedded in the material of choice and cocultured for one week, we were able to evaluate effects on vascular network formation (FIG. 5B). Visually, it is apparent that while no meaningful differences appear between networks grown in FBN gels or tFBN gels, there was no endothelial growth among the cells seeded in HDFGs, and thus no vascular network formation (HDFG: 1476±1591 μm², FBN gel: 266,000±98,600 μm², tFBN gel: 293,000±69,000 μm², both comparisons: p<0.0001, FIG. 5C, 5D). Indeed, a live/dead assay showed that ^{˜}99% of endothelial cells embedded in HDFGs were dead, likely due to the exposure to such high concentrations of thrombin. While fibroblasts were able to grow, the extent of their cellular growth and migration was significantly less than those of fibroblasts embedded in FBN or tFBN gels (HDFG: 352,000±206,000 μm², FBN gel: 753,000±113,000 μm², tFBN gel: 848,000±164,000 μm², HDFG vs FBN: p=, HDFG vs tFBN: p<0.0001, FIG. 5C, 5E). To determine cell-level effects, HUVECs were cultured for 16 hours on thin films of each material (5F). We can see that cells cultured on FBN and tFBN gels display more spreading and greater surface area, while those cultured on HDFG remained circular and small (5G). Quantification of cell attachment showed that cells attached significantly better to FBN gels and tFBN gels than HDFGs (HDFG: 4±5 cells/imaging frame, FBN: 15±8 cells/imaging frame, tFBN: 20±6 cells/imaging frame, HDFG vs FBN: p=0.0034, HDFG vs tFBN: p<0.0001, FIG. 5H). Cells grown on HDFG were smaller than those grown on FBN gel or tFBN gel (HDFG: 533±300 μm², FBN: 1855±703 μm², tFBN: 2256±1471 μm², HDFG vs FBN: p=0.0049, HDFG vs tFBN: p<0.0001, FIG. 5I). They also had a smaller perimeter (HDFG: 120±56 μm, FBN: 262±80 μm, tFBN: 286±75 μm, HDFG vs FBN: p<0.0001, HDFG vs tFBN: p<0.0001, FIG. 5J) and were more circular (HDFG: 0.54±0.23, FBN: 0.38±0.16, tFBN: 0.29±0.12, HDFG vs FBN: p=0.034, HDFG vs tFBN: p=0.0008, FIG. 5K). Overall, these results demonstrate the relative intolerance of HUVECs to growth into and onto HDFG, providing insight into the capsular formation witnessed in our in vivo studies.

Statistical analysis was performed using GraphPad Prism 9 software. Data was evaluated using a one-way ANOVA with Tukey's post hoc using a 95% confidence interval or an unpaired Student's t-test. Significance was defined as p<0.05. Where appropriate, outliers were excluded following ROUT evaluation (Q=1%).

### ADDITIONAL ASPECTS

1. A colloidal mixture, comprising:
fibrin nanoparticles;
a blood co-factor;
thrombin; and
a calcium salt.

2. The colloidal mixture according to any preceding aspect, wherein the blood co-factor comprises whole blood, plasma, fibrinogen/thrombin, or a combination thereof.

3. The colloidal mixture according to any preceding aspect, wherein the blood co-factor comprises platelet-poor plasma, platelet-rich plasma.

4. The colloidal mixture according to any preceding aspect, wherein the calcium salt comprises CaCl₂.

5. The colloidal mixture according to any preceding aspect, wherein the calcium salt is present at a concentration from 1-1,000 μM, from 5-1,000 μM, from 10-1,000 μM, from 25-1,000 μM, from 50-1,000 μM, from 100-1,000 μM, from 250-1,000 μM, from 1-500 μM, from 5-500 μM, from 10-500 μM, from 25-500 μM, from 50-500 μM, from 100-500 μM, from 250-500 μM, from 1-250 μM, from 5-250 μM, from 10-250 μM, from 25-250 μM, from 50-250 μM, from 100-250 μM, from 1-100 μM, from 5-100 μM, from 10-100 μM, from 25-100 μM, from 50-100 μM, from 1-10 μM, from 1-25 μM, from 5-50 μM, or from 25-75 μM.

6. The colloidal mixture according to any preceding aspect, wherein the thrombin is present at a concentration from 0.01-10 U/ml, from 0.05-10 U/ml, from 0.1-10 U/ml, from 0.5-10 U/ml, from 1-10 U/ml, from 0.01-2.5 U/ml, from 0.05-2.5 U/ml, from 0.1-2.5 U/ml, from 0.5-2.5 U/ml, from 1-2.5 U/ml, from 0.01-1.5 U/ml, from 0.05-1.5 U/ml, from 0.1-1.5 U/ml, from 0.5-1.5 U/ml, from 0.01-1 U/ml, from 0.1-1 U/ml, from 0.5-1 U/ml, from 0.01-0.75 U/ml, from 0.05-0.75 U/ml, from 0.1-0.75 U/ml, or from 0.25-0.75 U/ml.

7. The colloidal mixture according to any preceding aspect, wherein the fibrin nanoparticles comprise neonatal fibrin.

8. The colloidal mixture according to any preceding aspect, wherein the fibrin nanoparticles comprise cord blood fibrin.

9. The colloidal mixture according to any preceding aspect, wherein the fibrin nanoparticles are present at a concentration from 0.1-250 mg/ml, from 1-250 mg/ml, from 5-250 mg/ml, from 10-250 mg/ml, from 25-250 mg/ml, from 50-250 mg/ml, from 100-250 mg/ml, from 1-100 mg/ml, from 5-100 mg/ml, from 10-100 mg/ml, from 25-100 mg/ml, from 50-100 mg/ml, from 1-75 mg/ml, from 5-75 mg/ml, from 10-75 mg/ml, from 25-75 mg/ml, from 1-50 mg/ml, from 5-50 mg/ml, from 10-50 mg/ml, from 25-50 mg/ml, from 1-25 mg/ml, from 5-25 mg/ml, from 10-25 mg/ml, or from 20-40 mg/ml.

10. The colloidal mixture according to any preceding aspect, comprising the fibrin nanoparticles in an amount of 1-5,000M particles/ml, 1-1,000M particles/ml, 25-1,000M particles/ml, 50-1,000M particles/ml, 100-1,000M particles/ml, 250-1,000M particles/ml, 500-1,000M particles/ml, 1-500M particles/ml, 25-500M particles/ml, 50-500M particles/ml, 100-500M particles/ml, 250-500M particles/ml, 1-250M particles/ml, 25-250M particles/ml, 50-250M particles/ml, 100-250M particles/ml, 1-100M particles/ml, 25-100M particles/ml, 50-100M particles/ml, 1-50M particles/ml, 1-25M particles/ml, or 25-50M particles/ml.

11. The colloidal mixture according to any preceding aspect, wherein the fibrin nanoparticles have an average hydrodynamic diameter from 50-1,000 nm, from 50-500 nm, from 50-250 nm, from 100-500 nm, from 100-250 nm, from 100-300 nm, from 200-300 nm, from 250-350 nm, from 300-400 nm, from 250-500 nm, or from 500-1,000 nm.

12. The colloidal mixture according to any preceding aspect, further comprising a therapeutic agent.

13. The colloidal mixture according to any preceding aspect, wherein the therapeutic agent comprises an antibiotic, antifungal, analgesic, growth factor, cell therapy, or organoid.

14. The colloidal mixture according to any preceding aspect, wherein the therapeutic agent comprises a retinoid.

15. The colloidal mixture according to any preceding aspect, wherein the therapeutic agent comprises tazarotene.

16. The colloidal mixture according to a preceding aspect, wherein the growth factor comprises keratinocyte growth factor (KGF), platelet derived growth factor (PDGF), transforming growth factor-beta (TGFβ), interleukin, activin, colony stimulating factor, connective tissue growth factor (CTGF), epidermal growth factor (EGF), Epigen, erythropoietin, fibroblast growth factor (FGF), galectin, hepatoma-derived growth factor (HDGF), hepatocyte growth factor, insulin-like growth factor binding protein (IGFBP), insulin-like growth factor, insulin, leptin, macrophage migration inhibitory factor, melanoma inhibitory factor, myostatin, noggin, nephroblastoma overexpressed (NOV), omentin, oncostatinM, osteopontin, osteoprotogerin (OPG), periostin, placenta growth factor, placental lactogen, prolactin, RANK ligand, retinol binding protein, stem cell factor, transforming growth factor, and vascular endothelial growth factor (VEGF).

17. The colloidal mixture according to a preceding aspect, wherein the cell therapy comprises stem cell, T cell, a lymphocyte, a natural killer cell, bone marrow cell, hepatocyte, fibroblast, pancreatic islet cell, chondrocyte, keratinocyte, dendritic cell, macrophage, or combination thereof.

18. A method of making the colloidal mixture according to any preceding aspect, comprising combining fibrin nanoparticles with thrombin and a blood co-factor.

19. The method according to a preceding aspect, wherein the colloidal mixture is formed by combining lyophilized fibrin nanoparticles with composition comprising thrombin and a blood co-factor.

20. The method according to a preceding aspect, wherein the lyophilized fibrin nanoparticles, the composition, or both comprise a calcium salt.

21. The method according to a preceding aspect, further comprising the step of obtaining the blood co-factor from a subject.

22. The method according to a preceding aspect, further comprising obtaining blood from a subject, and separating plasma from the blood.

23. A method of treating a wound in a subject in need thereof, comprising contacting the wound with the colloidal mixture according to a preceding aspect.

24. A method of treating a wound in a subject in need thereof, comprising forming the colloidal mixture according to a preceding aspect, and contacting the wound with the colloidal mixture.

25. The method according to a preceding aspect, wherein the colloidal mixture is forming by combining fibrin nanoparticles with thrombin and a blood cofactor.

26. The method according to a preceding aspect, wherein the colloidal mixture is forming by combining lyophilized fibrin nanoparticles with an aqueous composition comprising thrombin, a calcium salt, and a blood cofactor.

27. The method according to a preceding aspect, wherein the blood co-factor is obtained from the subject.

28. The method according to any preceding aspect, wherein the wound is a surgical wound.

29. The method according to any preceding aspect, wherein the wound is a vascular wound, ocular wound, or a chronic wound.

30. A method for promoting healing following a surgical anastomosis, comprising administering the colloidal mixture according to any preceding aspect to an anastomotic site.

31. The method according to any preceding aspect, wherein the surgical anastomosis is a vascular anastomosis or intestinal anastomosis.

32. A method of surgically joining a first vessel and second vessel, comprising proximally configuring said first vessel and said second vessel in fluid communication, and applying the colloidal composition according to a preceding aspect to the proximal location.

33. The method according to any preceding aspect, wherein the first vessel and second vessel are proximally configured with sutures, adhesives, or a combination thereof.

34. A kit, comprising a first composition comprising fibrin nanoparticles, and a second composition comprising thrombin, a calcium salt, a blood co-factor, or a combination thereof.

35. The kit according to a preceding aspect, wherein the fibrin nanoparticles are lyophilized.

36. The kit according to a preceding aspect, further comprising a blood collecting system.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A colloidal mixture, comprising:
fibrin nanoparticles;
a blood co-factor;
thrombin; and
a calcium salt.

2. The colloidal mixture according to claim 1, wherein the blood co-factor comprises whole blood, plasma, fibrinogen/thrombin, or a combination thereof.

3. The colloidal mixture according to claim 1, wherein the blood co-factor comprises platelet-poor plasma, platelet-rich plasma.

4. The colloidal mixture according to claim 1, wherein the calcium salt comprises CaCl₂.

5. The colloidal mixture according to claim 1, wherein the calcium salt is present at a concentration from 1-1,000 μM.

6. The colloidal mixture according to claim 1, wherein the thrombin is present at a concentration from 0.01-10 U/ml.

7. The colloidal mixture according to claim 1, wherein the fibrin nanoparticles comprise neonatal fibrin or equine fibrin.

8. The colloidal mixture according to claim 1, wherein the fibrin nanoparticles comprise cord blood fibrin.

9. The colloidal mixture according to claim 1, wherein the fibrin nanoparticles are present at a concentration from 0.1-250 mg/ml.

10. The colloidal mixture according to claim 1, comprising the fibrin nanoparticles in an amount of 1-5,000M particles/ml.

11. The colloidal mixture according to claim 1, wherein the fibrin nanoparticles have an average hydrodynamic diameter from 50-1,000 nm.

12. The colloidal mixture according to claim 1, further comprising a therapeutic agent.

13. The colloidal mixture according to claim 12, wherein the therapeutic agent comprises an antibiotic, antifungal, analgesic, growth factor, cell therapy, or organoid.

14. A method of making the colloidal mixture according to claim 1, comprising combining fibrin nanoparticles with thrombin and a blood co-factor.

15. The method according to claim 14, wherein the colloidal mixture is formed by combining lyophilized fibrin nanoparticles with composition comprising thrombin and a blood co-factor.

16. The method according to claim 15, wherein the lyophilized fibrin nanoparticles, the composition, or both comprise a calcium salt.

17. A method of treating a wound in a subject in need thereof, comprising contacting the wound with the colloidal mixture according to claim 1.

18. A method of treating a wound in a subject in need thereof, comprising forming the colloidal mixture according to the method of claim 14, and contacting the wound with the colloidal mixture.

19. The method according to claim 18, wherein blood co-factor is obtained from the subject in need thereof.

20. A kit, comprising a first composition comprising fibrin nanoparticles, and a second composition comprising thrombin, a calcium salt, a blood co-factor, or a combination thereof.
